SEQUENCE LISTING (24 sequences)

SEQ ID NO: 1 - protein, 301 aa, Salmonella enterica
MIFSVQELSCGGKSMLSPTTRNMGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALALRTKAALEREQLPDCYFAMVELDIQRSSSECGIFSLALAKKLQLEFMNLVKIHEDNICERLCGEEPFLPSDKADRYLPVSFYKHTQGAQRLNEYVEANPAAGSSIVNKKNETLYERFDNNAVMLNDKKLSISAHKKRIAEYKSLLKP

SEQ ID NO: 2 - protein, 302 aa, Salmonella enterica
MIFSVQELSCGGKSMLSPTTRNMGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGAQRLNEYVEANPAAGSSIVNKKNETLYERFDNNAVMLNDKKLSISAHKKRIAEYKSLLKP

SEQ ID NO: 3 - protein, 302 aa, Salmonella enterica
MIFSVQELSCGGKSMLSPTTRNMGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVEANPAAGSSIVNKKNETLYERFDNNAVMLNDKKLSISAHKKRIAEYKSLLKS

SEQ ID NO: 4 - protein, 280 aa, Salmonella enterica
MGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVEANPAAGSSIVNKKNETLYERFDNNAVMLNDKKLSISAHKKRIAEYKSLLKS

SEQ ID NO: 5 - protein, 302 aa, Salmonella enterica; identical to SEQ ID NO: 3

SEQ ID NO: 6 - protein, 280 aa, Salmonella enterica
MGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVEANPAAGSSIVNKKNETLYERFDNNAVMLNDKKLSISAHKKRIAEYKSLLKP

SEQ ID NO: 7 - protein, 288 aa, Salmonella enterica
MLSPTTRNMGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVEANPAAGSSIVNKKNETLYERFDNNAVMLNDKKLSIFAHKKRIAEYKSLLKP

SEQ ID NO: 8 - protein, 288 aa, Salmonella enterica
MLSPTTRNMGASLSPQPDVSGELNTEALTCIVERLESEIIDGSWIHISYEETDLEMMPFLVAQANKKYPELNLKFVMSVHELVSSIKETRMEGVESARFLVNMGSSGIHISVVDFRVMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVQANPAAGSSIVNKKNETLYERFDNNAVMLNDKKLSISAHKKRIAEYKSLLKP

SEQ ID NO: 9 - protein, 288 aa, Salmonella enterica
MLSPTTRNMGASLSPQSDVSGELNTEALTCIVERLESEIIDGSWIHISYEETDLEMMPFLVAQANKKYPELNLKFVMSVHELVSSIKETRMEGVESARFIVNMGSSGIHVSVVDFRVMDGKTSVILFEPAACSAFGPALLALRTKAALEREQLPDCYFAMVELDIQRSSSECGIFSLALAKKLHLEFMNLVKIHEDNICERLCGEEPFLPSDKADRYLPVSFYKHTQGVQRLNEYVQANPAAGSSIVNKKNETLYERFDNNAVMLNDKKLSISAHKKRIAEYKSLLKP

SEQ ID NO: 10 - DNA, 906 bp, Salmonella enterica
atgatattttcggtgcaggagctatcatgtggagggaaaagtatgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgcactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgctatctgccggtgagtttttacaaacatactcaaggcgcacaacgattaaatgaatatgtggaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaaccgtaa

SEQ ID NO: 11 - DNA, 909 bp, Salmonella enterica
atgatattttcggtgcaggagctatcatgtggagggaaaagtatgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgctatctgccggtgagtttttacaaacatactcaaggcgcacaacgattaaatgaatatgtggaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaaccgtaa

SEQ ID NO: 12 - DNA, 909 bp, Salmonella enterica
atgatattttcggtgcaggagctatcatgtggagggaaaagtatgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgttatctgccggtgagtttttacaaacatactcaaggcgtacaacgattaaatgaatatgtggaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaatcgtaa

SEQ ID NO: 13 - DNA, 843 bp, Salmonella enterica
atgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgttatctgccggtgagtttttacaaacatactcaaggcgtacaacgattaaatgaatatgtggaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaatcgtaa

SEQ ID NO: 14 - DNA, 909 bp, Salmonella enterica; identical to SEQ ID NO: 12

SEQ ID NO: 15 - DNA, 843 bp, Salmonella enterica
atgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgctatctgccggtgagtttttacaaacatactcaaggcgtacaacgattaaatgaatatgtggaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaaccgtaa

SEQ ID NO: 16 - DNA, 867 bp, Salmonella enterica
atgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgcggtatttttagcctggcgctcgccaaaaaacttcagcttgaatttatgaacttagtaaaaattcatgaagataatatttgtgaacgtctgtgtggtgaagaaccttttctcccgtccgataaagcagaccgctatctgccggtgagtttttacaaacatactcaaggcgtacaacgattaaatgaatatgtggaggccaatccggcggcgggaagcagtatagtaaacaaaaagaatgaaacgctttatgagcgattcgataacaatgccgttatgctaaacgataaaaaactctctatattcgctcataaaaaaaggatagctgaatataagtctttacttaaaccgtaa

SEQ ID NO: 17 - DNA, 867 bp, Salmonella enterica
atgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttctcgtaaatatgggaagttcaggtatccatatttcagtcgtcgattttagagttatggacggaaagacatcggtgattttgttcgaaccagcagcgtgtagcgcttttggacctgctttactggcgttgaggaccaaagcagctcttgaacgtgaacaactgcctgattgttattttgctatggtcgagctggacattcaacgaagctcttctgaatgtggtatttttagcctggcgctcgccaaaaaacttcagcttgaatttatgaacttagtaaaaattcatgaagataatatttgtgaacgtctgtgtggtgaagaaccttttctcccgtctgataaagcagaccgttatctgccggtgagtttttacaaacatactcaaggcgtacaacgattaaatgaatatgtgcaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttgcttaaaccgtaa

SEQ ID NO: 18 - DNA, 867 bp, Salmonella enterica
atgctaagtcctacgactcgtaatatgggggcgagtttatcgcctcagtctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctataaaggagaccagaatggaaggcgttgaatctgcccgatttatcgtaaatatgggaagttcaggtatccatgtttcagtcgtcgattttagagttatggacggaaagacatcggtgattttgttcgaaccagcagcgtgtagcgcttttggacctgctttactggcattgaggaccaaagcagctcttgaacgtgaacaattgcctgattgttattttgctatggtcgagctggacattcaacgaagctcttctgaatgcggtatttttagcctggcgctcgccaaaaaacttcatcttgaatttatgaacttagtaaaaattcatgaagataatatttgtgaacgtctgtgtggtgaagaaccttttctcccgtccgataaagcagaccgctatctgccggtgagtttttacaaacatactcaaggcgtacaacgattaaatgaatatgtgcaggccaatccggcggcgggaagcagtatagtaaacaaaaagaatgaaacgctttatgagcgattcgataacaatgccgttatgctaaacgataaaaaactctctatatccgctcataaaaaaaggatagctgaatataagtctttacttaaaccgtaa

SEQ ID NO: 19 - protein, 302 aa, Artificial
  AvrA consensus
MIFSVQELSCGGKSMLSPTTRNMGASLSPQXDVSGELNTEALTCIVERLESEIIDGSWIHISYEETDLEMMPFLVAQANKKYPELNLKFVMSVHELVSSIKETRMEGVESARFXVNMGSSGIHXSVVDFRVMDGKTSVILFEPAACSAFGPAXLALRTKAALEREQLPDCYFAMVELDIQRSSSECGIFSLALAKKLXLEFMNLVKIHEDNICERLCGEEPFLPSDKADRYLPVSFYKHTQGXQRLNEYVXANPAAGSSIVNKKNETLYERFDNNAVMLNDKKLSIXAHKKRIAEYKSLLKX

SEQ ID NO: 20 - DNA, 24 bp, Artificial
  forward primer for IL-8
tgcataaagacatactccaaacct

SEQ ID NO: 21 - DNA, 22 bp, Artificial
  reverse primer for IL-8
aattctcagccctcttcaaaaa

SEQ ID NO: 22 - DNA, 21 bp, Artificial
  forward primer for GAPDH
cttcaccaccatggagaaggc

SEQ ID NO: 23 - DNA, 21 bp, Artificial
  reverse primer for GAPDH
ggcatggactgtggtcatgag

SEQ ID NO: 24 - protein, 9 aa, Artificial
  hemagglutinin tag
YPYDVPDYA

(19) United States
(12) Patent Application Publication
(10) Pub. No.: US 2012/0039798 A1
(43) Pub. Date: Feb. 16, 2012

(54) METHODS OF MODIFYING P53 ACETYLATION AND TREATING CANCER USING AVRA

(75) Inventor: Jun Sun, Rochester, NY (US)

(73) Assignee: UNIVERSITY OF ROCHESTER, Rochester, NY (US)

(21) Appl. No.: 13/254,664

(22) PCT Filed: Mar. 2, 2010
(86) PCT No.: PCT/US10/25876

§ 371 Date: Oct. 20, 2011

Related U.S. Application Data
(60) Provisional application No. 61/156,718, filed on Mar. 2, 2009.

(51) Int. Cl. A61K 51/00; A61K 38/48; A61K 39/00; A61P 35/00; A61P 35/02; C12N 5/09; A61K 48/00

(52) U.S. Cl. 424/1.11; 435/375; 424/94.65; 514/44 R; 424/184.1

(57) ABSTRACT

The present invention relates to methods and compositions for the treatment of cancer. The methods and compositions involve the use of the Salmonella effector protein AvrA, as well as variants or fragments thereof, or encoding nucleic acids. The AvrA effector protein is demonstrated to enhance p53 acetylation, disrupt the cell cycle progression in treated cells, and enhance the killing of cancer cells. In this way, the methods and compositions can treat cancerous conditions either alone or in combination with other therapies.

This application claims the benefit of U.S. Provisional Patent Application Ser. No. 61/156,718, filed Mar. 2, 2009, which is hereby incorporated by reference in its entirety.

This invention was made with government support under grant number KO1 DK075386 awarded by the National Institutes of Health. The government has certain rights in this invention.

### FIELD OF THE INVENTION

The present invention generally relates to methods, preparations and pharmaceutical compositions for treating cancer in mammalian subjects.

### BACKGROUND OF THE INVENTION

Salmonella is a well-armed opportunistic pathogen that produces a diverse array of pathogenic factors and causes infection. It uses a type three secretion system (“TTSS”), a needle system which injects bacterial pathogenic proteins into host cells (Lane, “p53, Guardian of the Genome,” Nature 358:15-16 (1992); Patel et al., “The Functional Interface Between Salmonella and its Host Cell: Opportunities for Therapeutic Intervention,” Trends in Pharmacological Sciences 26:564-570 (2005)). The virulence proteins injected by the TTSS are called effectors. AvrA is a newly-described Salmonella effector translocated into host cells (Hardt et al., “A Secreted Salmonella Protein with Homology to an Avirulence Determinant of Plant Pathogenic Bacteria,” Proc. Natl. Acad. Sci. USA 94:9887-9892 (1997)). Recent studies have shown that the AvrA gene is present in 80% of Salmonella enterica serovar (Streckel et al., “Expression Profiles of Effector Proteins SopB, SopD1, SopE1, and AvrA Differ with Systemic, Enteric, and Epidemic Strains of Salmonella enterica,” Molecular Nutrition & Food Research 48:496-503 (2004)). AvrA belongs to a family of cysteine proteases which regulates diverse bacterial-host interactions (Collier-Hyams et al. “Cutting Edge: Salmonella AvrA Effector Inhibits the Key Proinflammatory, Anti-apoptotic NF-kappa B Pathway,” J. Immunol. 169:2846-2850; Ye et al., “Salmonella Effector AvrA Regulation of Colonic Epithelial Cell Inflammation by Deubiquitination,” Am. J. Pathol. 171:882-892 (2007); and Jones et al., “Salmonella AvrA Coordinates Suppression of Host Immune and Apoptotic Defenses via JNK Pathway Blockade,” Cell Host & Microbe 3:233-244 (2008)). Other family members related to AvrA include the adenovirus protease AVP, Yersinia virulence factor YopJ (Yersinia outer protein J), and the Xanthomonas campestris pv. vesicatoria protein AvrBsT (Orth et al., “Disruption of Signaling by Yersinia Effector YopJ, a Ubiquitin-Like Protein Protease,” Science 290:1594-1597 (2000)). Several of these bacterial effectors mimic the activity of a eukaryotic protein and debilitate their target cells.

The p53 protein is known as a “guardian of the genome” because of its crucial role in coordinating cellular responses to genotoxic stress (Lane, “p53, Guardian of the Genome,” Nature 358:15-16 (1992); Levine, “p53, The Cellular Gatekeeper for Growth and Division,” Cell 88:323-331 (1997); Melino et al., “p73: Friend or Foe in Tumorigenesis,” Nature Reviews 2:605-615 (2002); Harris et al., “The p53 Pathway: Positive and Negative Feedback Loops,” Oncogene 24:2899-2908 (2005); and Kruse et al., “SnapShot: p53 Posttranslational Modifications,” Cell 133:930-930 e931 (2008)). The tumor suppression effects of p53 are mediated by a variety of mechanisms, including cell cycle arrest, apoptosis, and cellular senescence (Vogelstein et al., “Surfing the p53 Network,” Nature 408:307-310 (2000)). The p53 protein is tightly regulated, so that its protein product usually exists in a latent form, and at low levels in unstressed cells. However, the steady-state levels and transcriptional activity of p53 increase dramatically in cells that undergo various types of stress (Vousden et al., “Live or Let Die: The Cell's Response to p53,” Nature Reviews 2:594-604 (2002)). Although the precise mechanisms of p53 activation are not fully understood, they involve posttranslational modification, including ubiquitination, acetylation, phosphorylation, sumoylation, neddylation, methylation, and glycosylation of the p53 polypeptide (Kruse et al., “SnapShot: p53 Posttranslational Modifications,” Cell 133:930-930 e931 (2008); and Vousden et al., “Live or Let Die: The Cell's Response to p53,” Nature Reviews 2:594-604 (2002)).

Microbial infection is a stress to the host. Virus and mycoplasma are known to be involved in the p53 pathway (Levine, “p53, The Cellular Gatekeeper for Growth and Division,” Cell 88:323-331 (1997); Hsu et al., “HCMV IE2-Mediated Inhibition of HAT Activity Downregulates p53 Function,” EMBO J. 23:2269-2280 (2004); Nakamura et al., “Inhibition of p53 Tumor Suppressor by Viral Interferon Regulatory Factor,” J. Virology 75:7572-7582 (2001); Mauser et al., “The Epstein-Barr Virus Immediate-Early Protein BZLF1 Regulates p53 Function Through Multiple Mechanisms,” J. Virology 76:12503-12512 (2002); de la Cruz-Hernandez et al., “The Effects of DNA Methylation and Histone Deacetylase Inhibitors on Human Papillomavirus Early Gene Expression in Cervical Cancer, an in vitro and Clinical Study,” Virology 4:18 (2007); and Hoshino et al., “Role of Histone Deacetylase Inhibitor in Adenovirus-Mediated p53 Gene Therapy in Esophageal Cancer,” Anticancer Research 28:665-671 (2008); Logunov et al., “Mycoplasma Infection Suppresses p53, Activates NF-κB and Cooperates with Oncogenic Ras in Rodent Fibroblast Transformation,” Oncogene 27:4521-4531 (2008)). Mycoplasma infection plays the role of a p53-suppressing oncogene that cooperates with Ras in cell transformation (Logunov et al., “Mycoplasma Infection Suppresses p53, Activates NF-kappaB and Cooperates with Oncogenic Ras in Rodent Fibroblast Transformation,” Oncogene 27:4521-4531 (2008)). It is unknown whether and how Salmonella infection is involved in the posttranslational modification of p53. Although the exact function and mechanism of AvrA is not entirely clear, it is known that AvrA influences eukaryotic cell pathways that utilize ubiquitin (Ye et al., “Salmonella Effector AvrA Regulation of Colonic Epithelial Cell Inflammation by Deubiquitination,” Am. J. Pathol. 171:882-892 (2007)) and acetylation (Jones et al., “Salmonella AvrA Coordinates Suppression of Host Immune and Apoptotic Defenses via JNK Pathway Blockade,” Cell Host & Microbe 3:233-244 (2008)).

It would be desirable to determine whether AvrA and its homologs are capable of modulating the activity of p53, and in particular whether modulation of p53 can arrest cell cycle progression in cancer cells or alter the survival of healthy cells. The present invention is directed to overcoming these and other deficiencies in the art.

### SUMMARY OF THE INVENTION

A first aspect of the present invention relates to a method for inhibiting cancer cell proliferation that includes introducing into a cancer cell (i) an isolated AvrA protein or polypeptide fragment thereof or (ii) a nucleic acid molecule encoding the isolated AvrA protein or polypeptide fragment, wherein said introducing is effective for inhibiting cell cycle progression of the cancer cell. By inhibiting cell cycle progression, cancer cell proliferation can be inhibited.

A second aspect of the present invention relates to a method of treating a patient for cancer that includes administering to a patient having cancer a therapeutically effective dose of (i) an isolated AvrA protein or polypeptide fragment thereof, or (ii) a nucleic acid molecule encoding the AvrA protein or polypeptide fragment. This aspect of the invention includes inhibiting cancer cell proliferation as well as killing cancer cells.

A third aspect of the present invention relates to a therapeutic system that includes: a single unit dose including, in a pharmaceutically acceptable carrier, either (i) a therapeutically effective amount of an isolated AvrA protein or polypeptide fragment thereof, or (ii) a therapeutically effective amount of a nucleic acid molecule encoding the AvrA protein or polypeptide fragment; and a single unit dose including, in a pharmaceutically acceptable carrier, a therapeutically effective amount of an immunotherapeutic agent, chemotherapeutic agent or radiation therapeutic agent.

A fourth aspect of the present invention relates to a pharmaceutical composition that includes: a pharmaceutically acceptable carrier; a therapeutically effective amount of an isolated AvrA protein or polypeptide fragment thereof; and a therapeutically effective amount of an immunotherapeutic agent, chemotherapeutic agent, or radiation therapeutic agent.

The results presented in the accompanying Examples demonstrate that Salmonella AvrA is capable of modifying the expression levels of p53 and inducing p53 acetylation. Inactivation of p53 functions has been well documented as a common mechanism for tumorigenesis (Vogelstein et al., “Surfing the p53 Network,” Nature 408:307-310 (2000), which is hereby incorporated by reference in its entirety). Many cancer therapy drugs have been designed based on either reactivating p53 functions or inactivating p53 negative regulators. Since p53 is strongly activated in response to DNA damage, mainly through attenuation of the Mdm2-mediated negative regulatory pathway (Maya et al., “ATM-Dependent Phosphorylation of Mdm2 on Serine 395: Role in p53 Activation by DNA Damage,” Genes Dev. 15:1067-1077 (2001), which is hereby incorporated by reference in its entirety), many DNA damage-inducing drugs such as etoposide are very effective antitumor drugs in cancer therapy (reviewed in Chresta et al., “Oddball p53 in Testicular Tumors,” Nat. Med. 2:745-746 (1996); Lutzker et al., “A Functionally Inactive p53 Protein in Teratocarcinoma Cells is Activated by Either DNA Damage or Cellular Differentiation,” Nat. Med. 2:804-810 (1996), each of which is hereby incorporated by reference in its entirety).

AvrA is quite unique in that it can activate the p53 pathway while inhibiting the NF-κB pathway. The p53 pathway is inactive in almost all kinds of tumors and the NF-κB pathway is active in the tumor cells. Use of AvrA to activate p53 and inhibit NF-κB in cancer cells should inhibit proliferation and growth of cancer cells, and even induce cancer cell death. AvrA has been demonstrated to induce cell death in the HeLa cancer cell line.

In contrast to the effect of AvrA on cancer cells, AvrA should help to protect normal cells during chemotherapy, immunotherapy, or radiation therapy co-administration. In vitro, AvrA expression induces cell cycle arrest with increased cell numbers at the G0/G1 phase, and decreased cell numbers at the G2/M phase in normal host cells. In an in vivo animal model using normal (healthy) animals, AvrA appears to increase cell proliferation. Thus, the various aspects of the invention also include the protection of healthy cells during cancer treatment. Without being bound by belief, it is believed that AvrA, as an enzyme with dual activities, selectively activates pathways controlling cell fate in both normal and cancer cells. This would account for AvrA producing one result in cancer cells and a different result in normal cells.

Thus, the use of AvrA alone or in combination with one or more DNA damage agents is specifically contemplated. The combination can involve co-administration as well as administration in the form of a single formulation. Other features and advantages of the invention will be apparent from the following detailed description and claims.

### BRIEF DESCRIPTION OF THE DRAWINGS

FIGS. 1A-B illustrate a ClustalW multiple sequence alignment of nine exemplary AvrA amino acid sequences. SEQ ID NO: 1=Salmonella enterica serovar Typhimurium (Genbank Accession No. AAB83970, which is hereby incorporated by reference in its entirety); SEQ ID NO: 2=Salmonella typhimurium LT2 (Genbank Accession No. AAL21745, which is hereby incorporated by reference in its entirety); SEQ ID NO: 3=Salmonella enterica subsp. enterica serovar Gallinarum str. 287/91 (Genbank Accession No. CAR38577, which is hereby incorporated by reference in its entirety); SEQ ID NO: 4=Salmonella enterica subsp. enterica serovar Heidelberg str. SL486 (Genbank Accession No. EDZ24776, which is hereby incorporated by reference in its entirety); SEQ ID NO: 5=Salmonella enterica subsp. enterica serovar Enteritidis str. P125109 (Genbank Accession No. CAR34285, which is hereby incorporated by reference in its entirety); SEQ ID NO: 6=Salmonella enterica subsp. enterica serovar Kentucky str. CVM29188 (Genbank Accession No. EDX43702, which is hereby incorporated by reference in its entirety); SEQ ID NO: 7=Salmonella enterica subsp. enterica serovar Saintpaulia, strain SARA23 (Genbank Accession No. EDZ12687, which is hereby incorporated by reference in its entirety); SEQ ID NO: 8=Salmonella enterica subsp. enterica serovar Agona strain SL483 (Genbank Accession No. ACH48766, which is hereby incorporated by reference in its entirety); and SEQ ID NO: 9=Salmonella enterica subsp. enterica serovar Schwarzengrund str. CVM19633 (Genbank Accession No. ACF92027, which is hereby incorporated by reference in its entirety). Symbols: “*” denotes absolutely conserved residues; “:” and “.” denote conserved and semi-conserved substitutions, respectively.

FIGS. 2A-E illustrate a Dialign multiple sequence alignment of nine exemplary avrA open reading frames (DNA sequences). SEQ ID NO: 10=Salmonella enterica serovar Typhimurium (Genbank Accession No. AF013573, which is hereby incorporated by reference in its entirety); SEQ ID NO: 11=Salmonella typhimurium LT2 (Genbank Accession No. AE006468, which is hereby incorporated by reference in its entirety); SEQ ID NO: 12=Salmonella enterica subsp. enterica serovar Gallinarum str. 287/91 (Genbank Accession No. AM933173, which is hereby incorporated by reference in its entirety); SEQ ID NO: 13=Salmonella enterica subsp. enterica serovar Heidelberg str. SL486 (Genbank Accession No. ABEL01000005, which is hereby incorporated by reference in its entirety); SEQ ID NO: 14=Salmonella enterica subsp. enterica serovar Enteritidis str. P125109 (Genbank Accession No. AM933172, which is hereby incorporated by reference in its entirety); SEQ ID NO: 15=Salmonella enterica subsp. enterica serovar Kentucky str. CVM29188 (Genbank Accession No. ABAK02000001, which is hereby incorporated by reference in its entirety); SEQ ID NO: 16=Salmonella enterica subsp. enterica serovar Saintpaulia, strain SARA23 (Genbank Accession No. ABAN01000004, which is hereby incorporated by reference in its entirety); SEQ ID NO: 17=Salmonella enterica subsp. enterica serovar Agona strain SL483 (Genbank Accession No. CP001138, which is hereby incorporated by reference in its entirety); and SEQ ID NO: 18=Salmonella enterica subsp. enterica serovar Schwarzengrund str. CVM19633 (Genbank Accession No. CP001127, which is hereby incorporated by reference in its entirety). Symbols: “*” denotes absolutely conserved nucleic acids; “+” denotes conserved nucleic acids among subset of sequence aligned.

FIGS. 3A-C show that Salmonella but not TNFα increases p53 acetylation in the host cells. FIG. 3A shows the acetylation of p53 in human epithelial HCT116 and HCT116p53−/− cell lines. Cells were treated with TNF (10 ng/ml) for 30 minutes or incubated with S. typhimurium for 30 minutes, washed, incubated in fresh DMEM for 1 hour. Total cell lysates were analyzed for acetylated p53 and total p53 levels by immunoblot. FIG. 3B shows that Salmonella increases the acetylation of p53 in IEC-18 and MEF cells. Cells were treated with TNF (10 ng/ml) for 30 minutes or incubated with S. typhimurium for 30 minutes, washed, incubated in fresh DMEM for 1 hour. Total cell lysates were analyzed for acetylated p53, total p53, or β-actin levels by immunoblot. FIG. 3C shows the location of acetylated p53 in the intestinal epithelial cells using immunofluorescence staining Data are from a single experiment and are representative of >3 separate experiments.

FIGS. 4A-E show that Salmonella type three secretion effector AvrA is involved in the p53 acetylation. FIG. 4A shows AvrA protein expression in the indicated Salmonella strains. FIG. 4B shows the status of AvrA mRNA expression in normal intestinal epithelial cells infected with Salmonella by PCR. AvrA DNA is 1300 bp. GAPDH (425 bp) is an internal control for PCR. FIG. 4C shows that bacterial effector protein AvrA modulates p53 acetylation in epithelial cells. Intestinal epithelial IEC-18 cells were infected with TNFα, wild-type S. typhimurium (SL), mutant PhoP^{C}, AvrA-(without AvrA expression), or AvrA−/+(AvrA gene restored) for 30 minutes. Total cell lysates were analyzed for protein levels by immunoblot. FIG. 4D shows that HeLa were infected with PhoP^{C}, AvrA+ (AvrA overexpression), and AvrA− (without AvrA expression) for 30 minutes. Total cell lysates were analyzed for protein levels by immunoblot. FIG. 4E shows HCT116 p53−/− cells were transfected with the AvrA, p53 or AvrA +p53 plasmids for 24 hours. Lipo: LipofectAMINE2000; Empty: p-CMV-myc plasmid; AvrA: pCMV-myc-AvrA; and P53: pCMV-HA-p53. Total cell lysates were analyzed for protein levels by immunoblot.

FIGS. 5A-B show that Salmonella AvrA physically binds to p53, as measured by immunoprecipitation results using cell lysates from epithelial cells that were co-transfected with HA-p53 and pCMV-myc-AvrA or AvrA mutant C186A for 24 hours. FIG. 5A shows the immunoprecipitation performed with anti-HA antibody and total cell lysates analyzed for c-myc-AvrA and HA-p53 protein levels by immunoblot. FIG. 5B shows the immunoprecipitation performed with anti-HA antibody and total cell lysates analyzed for acetylated p53 and total p53 protein levels by immunoblot.

FIGS. 6A-B show the results of an in vitro AvrA transacetylase assay. FIG. 6A shows the scheme of the Salmonella AvrA mutants used in this study. FIG. 6B shows that AvrA functions as an acetyltransferase to acetylate p53. Transacetylase p300-CBP-associated factor (PCAF) was used as a positive control. AvrA protein at different concentration (0.15 μg-5 μg) was mixed with p53 in the reaction buffer. The reaction mixture was incubated at 30° C. for 30 minutes and stopped by the addition of an equal volume of SDS-gel sample buffer, denatured for 5 min at 95° C., then subjected to electrophoresis on SDS-PAGE, followed by immunoblot for detection. Data are from a single experiment and are representative of >3 separate experiments.

FIGS. 7A-B show that AvrA changes the p53 transcriptional activity and target genes. FIG. 7A shows that AvrA expression activates p53 transcriptional activity.

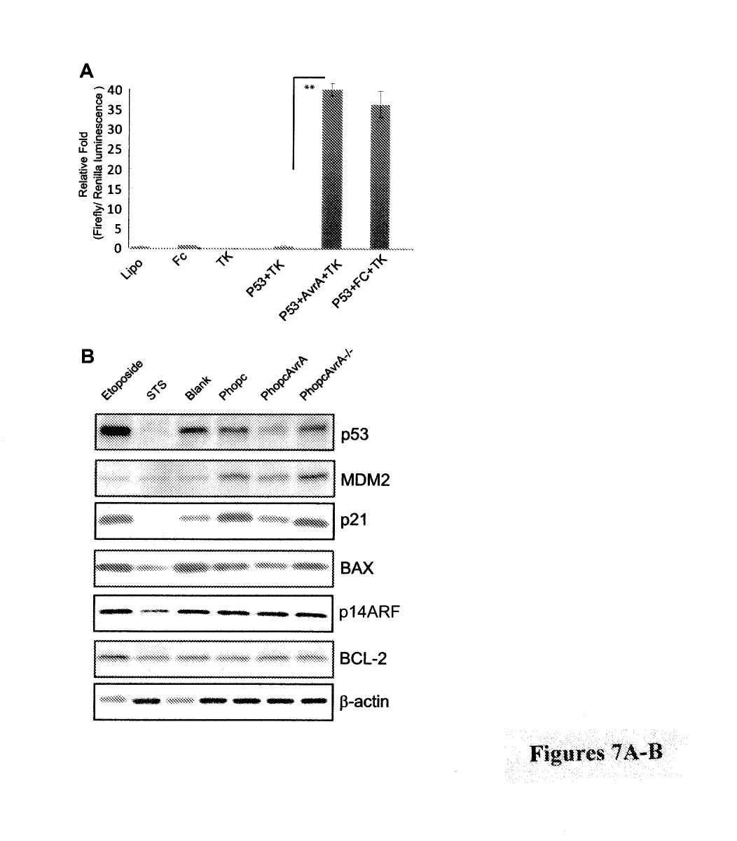

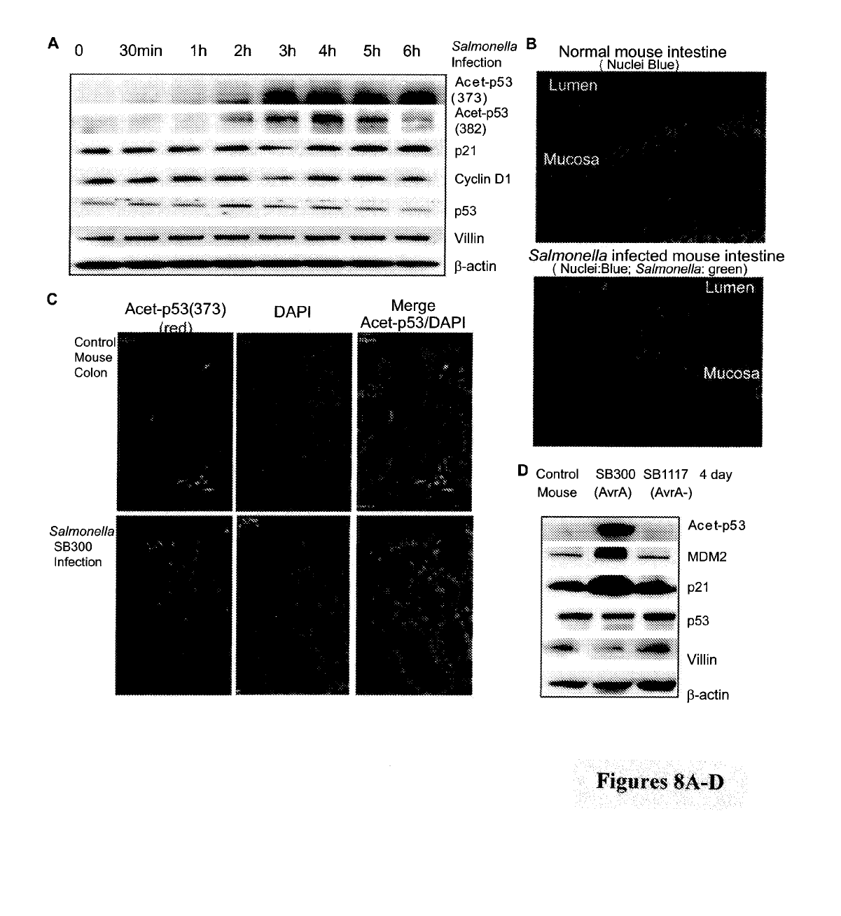

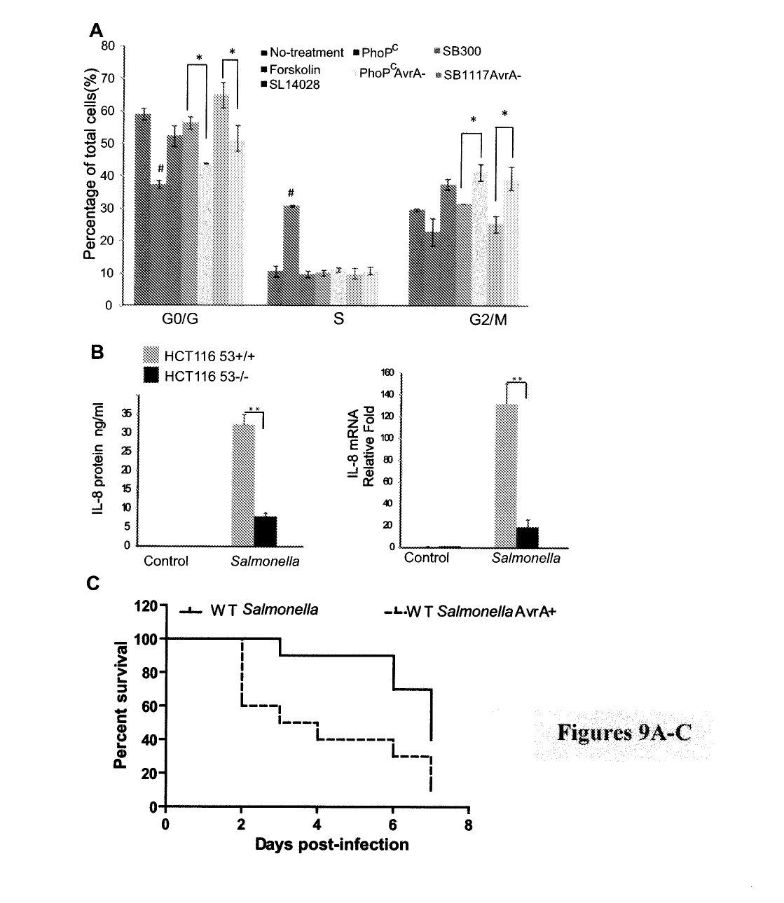

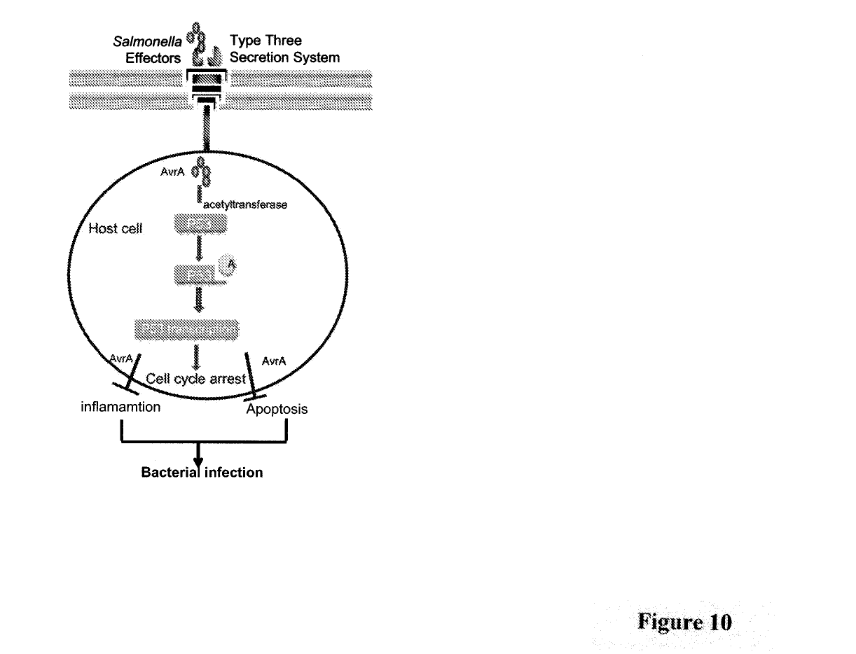

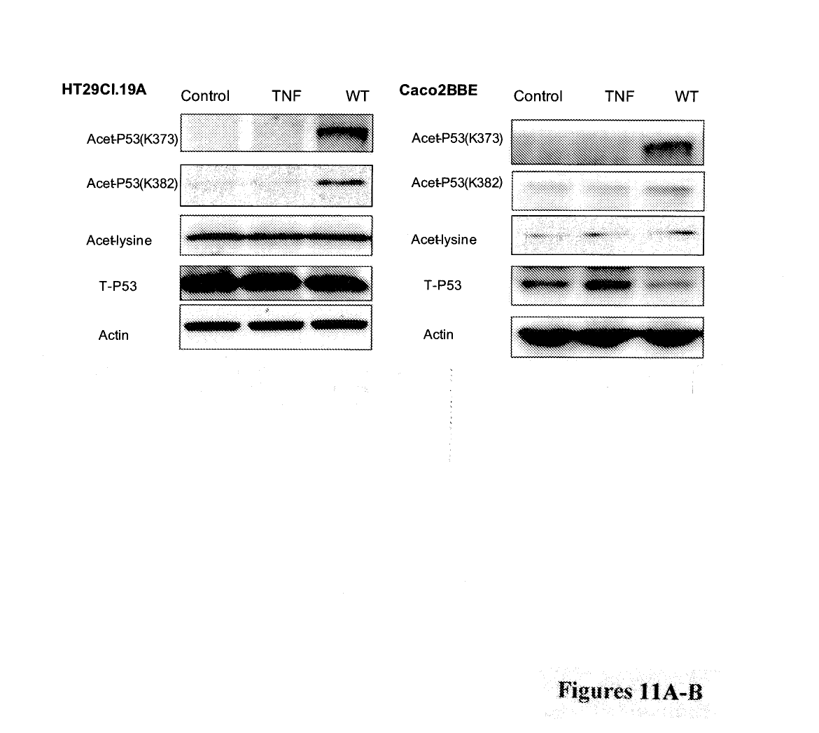

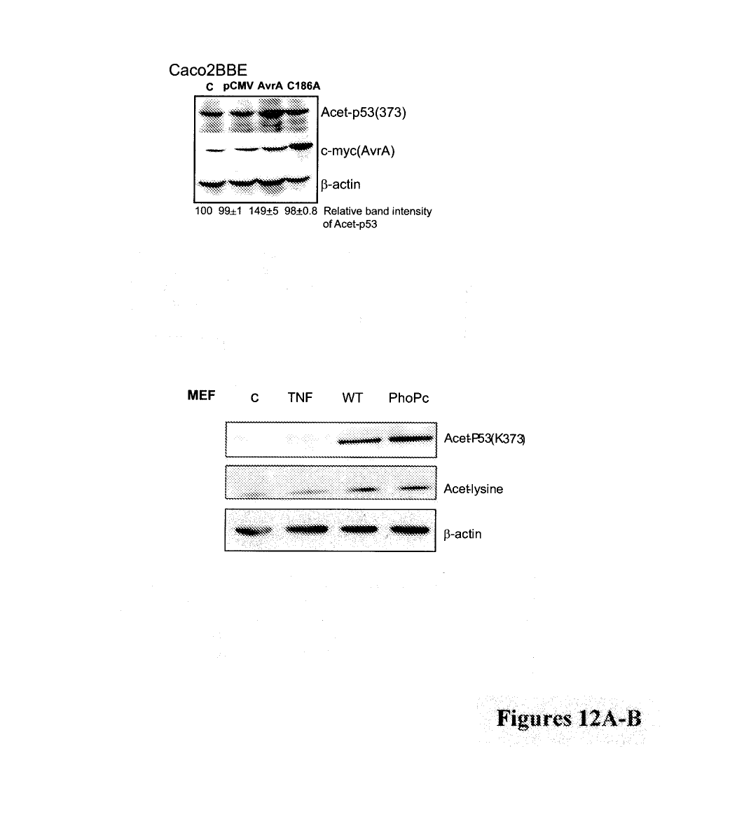

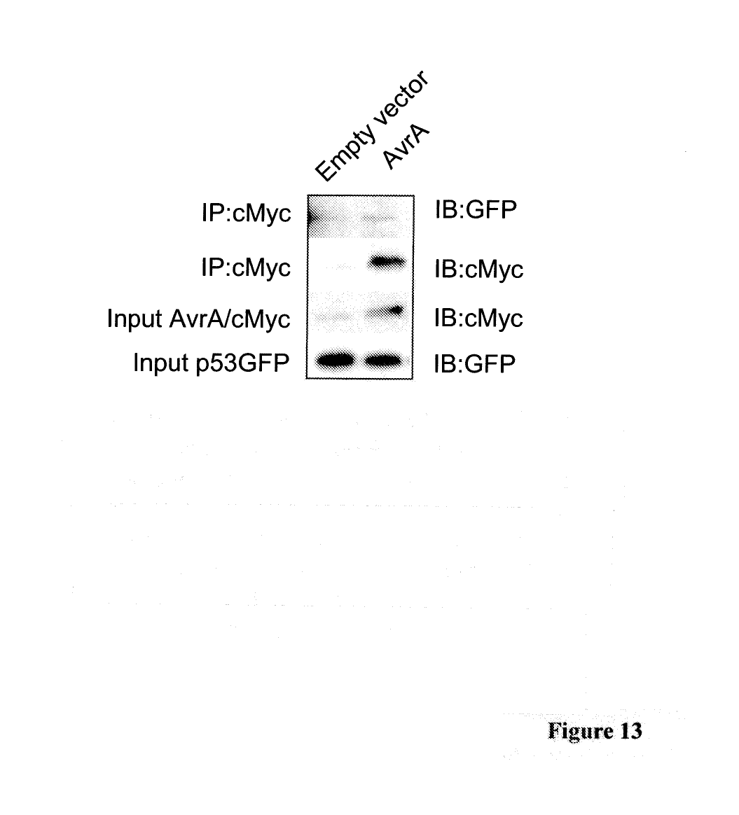

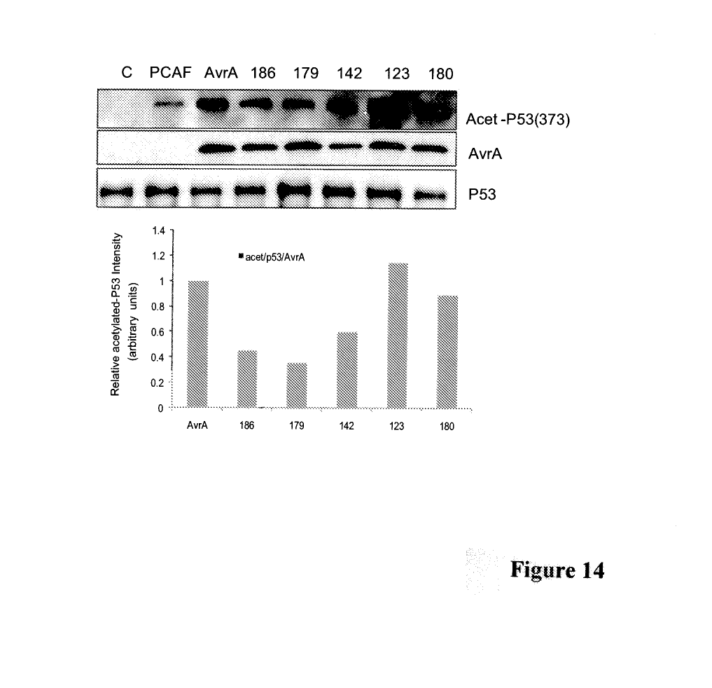

HCT116p53−/− cells were grown in 12-well plates in triplicates. Cells were treated with LipofectAMINE2000 (Lipo) or transfected with a pFC-p53 plasmid (Fc), a control plasmid pRL-TK(TK), a p53-Luc-cis reporter plasmid cotransfected with plasmid pRL-TK(p53+TK), a c-myc-AvrA cotransfected a p53-Luc-cis reporter and a pRL-TK plasmid (p53+AvrA+TK), or a p53-Luc-cis reporter(p53), a pFC-p53 plasmid, and a pRL-TK(p53+Fc+TK) using LipofectAMINE. After transfection for 24 h, cells were lysed, and luciferase activity was determined using the Dual Luciferase Reporter Assay System. Firefly luciferase activity was normalized to Renilla luciferase activity, and the activity was expressed as relative luminescence units. Data are from a single experiment and are representative of 3 separate experiments. **P53+TK (without AvrA) group vs. P53+AvrA+TK (AvrA sufficient) group, P<0.01. The p53+Fc+TK group is the positive control known to activate the p53 transcription activity. FIG. 7B shows that HeLa cells were treated with 1 μm STS (staurosporine), or 100 μm Etoposide 6 hours before harvest, or infected with PhoP^{C}, PhoP^{C}AvrA+/+, PhoP^{C}AvrA−/− for 30 minutes, washed, incubated in DMEM for another 6 hours before harvest. Proteins from total cell lysates were separated on 12% SDS-PAGE gels for Western blot analysis.

FIGS. 8A-D show that AvrA changes the acetylation of p53 and target genes of the p53 pathway in vivo. FIG. 8A shows that wild-type strain S. typhimurium ATCC14028 increased the acetylation of p53 post infection in mice post-infection 1 hour to 6 hours. FIG. 8B shows immunostaining of S. typhimurium (green) in the mouse colon. Bacteria were found in the mouse mucosa infected with S. typhimurium by day 4 post infection. FIG. 8C shows the location of p53 acetylation in the mouse large intestine after S. typhimurium infection. Enhanced nuclear staining of the acetylated p53 (red) was observed in the mouse colon with S. typhimurium infection. FIG. 8D shows that bacterial protein effector AvrA modulates the acetylated p53 expression in mouse colonic epithelial cells. Mice were infected with wild-type S. typhimurium SB300 with AvrA protein expression or SB1117 (AvrA gene mutant) for 4 days. Colonical epithelial cells were harvested, and total cell lysates were analyzed for acetylated p53 by immunoblot.

FIGS. 9A-C illustrate the cell physiological effects of AvrA for in vitro and in vivo models. FIG. 9A shows that AvrA expression increases cell numbers at G0/G1 phase, and decreases cell numbers at G2/M phase. Rat intestinal epithelial IEC18 cells were infected with bacteria for 30 min, washed and incubated in DMEM for 4 hours. Experimental groups: normal IEC18 cells; positive control Forskolin; SL14028 with insufficient AvrA; PhoP^{c} with AvrA expression, PhoP^{c} AvrA−; SB300: wild-type Salmonella 1344 with sufficient AvrA; SB1117: AvrA− mutant derived from SL1344; * AvrA sufficient group vs. AvrA deficient group, P<0.01, #AvrA sufficient or deficient group vs. Forskolin group, P<0.01. The single experiment was assayed in triplicate. Data are the means±SD of three separate experiments. FIG. 9B shows IL-8 protein and mRNA levels in the HCT116 and HCT116p53−/− cells after Salmonella infection. Cells were cultured in DMEM, followed by Salmonella-containing HBSS (1.6×10¹⁰ bacteria/ml) for 30 min, washed 3 times in HBSS, and incubated at 37° C. for 6 hours. Cell supernatants were removed and assayed for IL-8 by ELISA. Total RNA was extracted for real-time PCR. FIG. 9C shows the survival rate of mice post Salmonella typhimurium infection. Mice were infected with wildtype S. typhimurium strain 14028s (WT) with insufficient AvrA expression or WT 14028s with AvrA overexpression (WTAvrA+) for over 7 days. If a mouse showed indication that it had aspirated fluid or significant body weight loss (10% or more), and did not die immediately, the mouse was humanely euthanized. The protocol was approved by the University of Rochester University Committee on Animal Resources (UCAR).

FIG. 10 illustrates the working model of AvrA/p53 interaction during intestinal Salmonella infection (i.e., in otherwise normal cells).

FIGS. 11A-B illustrate that Salmonella but not TNF increases p53 acetylation in the host cells. The acetylation of p53 in human epithelial HT29C.19A (FIG. 11A) and Caco2BBE (FIG. 11B) cell lines is shown. Cells were treated with TNF (10 ng/ml) for 30 minutes or incubated with S. typhimurium for 30 minutes, washed, incubated in fresh DMEM for 1 hour. Total cell lysates were analyzed for acetylated p53, Lysine, and total p53 levels by immunoblot.

FIG. 12A shows that overexpression of AvrA increases p53 acetylation in epithelial cells. Intestinal epithelial Caco2BBE cells were transfected with pCMV-myc-AvrA or AvrA mutant C186A for 24 hours. Total cell lysates were analyzed for protein levels by immunoblot. Data are from a single experiment and are representative of >3 separate experiments. FIG. 12B shows that AvrA is involved in the p53 acetylation. MEF cells were infected with S. typhimurium (with low AvrA expression) and PhoPC (with high AvrA overexpression) for 30 minutes. Total cell lysates were analyzed for protein levels by immunoblot.

FIG. 13 shows that AvrA interacts with p53. Green fluorescent protein (GFP)-p53 was cotransfected with c-myc-AvrA in HeLa cells for 24 hours. Immunoprecipitation was performed with anti-c-Myc antibody. Total cell lysates were analyzed for c-myc-AvrA and GFP-p53 protein levels by immunoblot. Immunoblot with c-myc showed AvrA interacts with p53.

FIG. 14 illustrates the in vitro transacetylase assay of AvrA and its mutants. The wild-type AvrA, AvrA(C186A), AvrA(R180G), AvrA(C179A), AvrA(E142A), or AvrA(H123A) mutant proteins were purified from E. coli strain BL21(DE3). Purified wild-type p53 protein was used as substrate. Data are from a single experiment and are representative of >3 separate experiments.

### DETAILED DESCRIPTION OF THE INVENTION

The present invention relates to novel uses of the bacterial avirulence protein known as AvrA, as well as active polypeptide fragments thereof, and isolated nucleic acid molecules or expression vectors encoding the same. In particular, as noted above, the applicant has surprisingly demonstrated that AvrA is capable of inducing increased expression and acetylation of p53. The present invention discloses methods and compositions for treating cancer, for causing cell cycle arrest in cancer cells, inhibiting cancer cell proliferation, and killing cancer cells.

The types of cancer that can be treated according to the present invention include, without limitation, cancers of mescenchymal origin (sarcomas); cancers of epithelial origin (carcinomas); brain cancers; leukemias; and lymphomas. Non-limiting examples include leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

As used herein, the term “AvrA” is intended to encompass any AvrA homolog (including YopJ), but preferably Salmonella AvrA homologs. A consensus sequence of Salmonella AvrA is provided in SEQ ID NO: 19 as follows:

MIFSVQELSCGGKSMLSPTTRNMGASLSPQXDVSGELNTEALTCIVERLESEIIDGSWI
HISYEETDLEMMPFLVAQANKKYPELNLKFVMSVHELVSSIKETRMEGVESARFXVNMG
SSGIHXSVVDFRVMDGKTSVILFEPAACSAFGPAXLALRTKAALEREQLPDCYFAMVEL
DIQRSSSECGIFSLALAKKLXLEFMNLVKIHEDNICERLCGEEPFLPSDKADRYLPVSF
YKHTQGXQRLNEYVXANPAAGSSIVNKKNETLYERFDNNAVMLNDKKLSIXAHKKRIAE
YKSLLKX

where the consensus preferably comprises amino acids 23-302 of SEQ ID NO: 19, or alternatively amino acids 15-302 of SEQ ID NO: 19 or amino acids 1-302 of SEQ ID NO: 19, with X at position 31 being any amino acid, but preferably P or S; X at position 114 being any amino acid, but preferably L or I; X at position 124 being any amino acid, but preferably I or V; X at position 153 being optional or any amino acid, but preferably L; X at position 198 being any amino acid, but preferably Q or H; X at position 243 being any amino acid, but preferably A or V; X at position 251 being any amino acid, but preferably E or Q; X at position 287 being any amino acid, but preferably S or F; and X at position 302 being any amino acid, but preferably P or S.

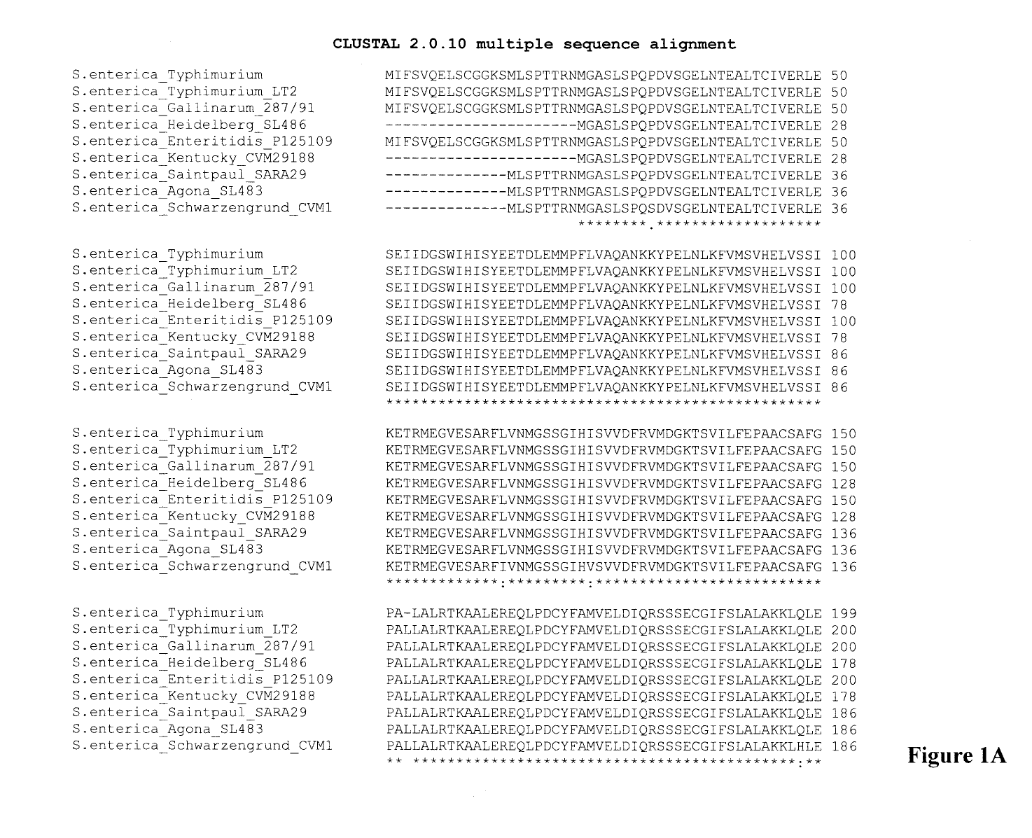

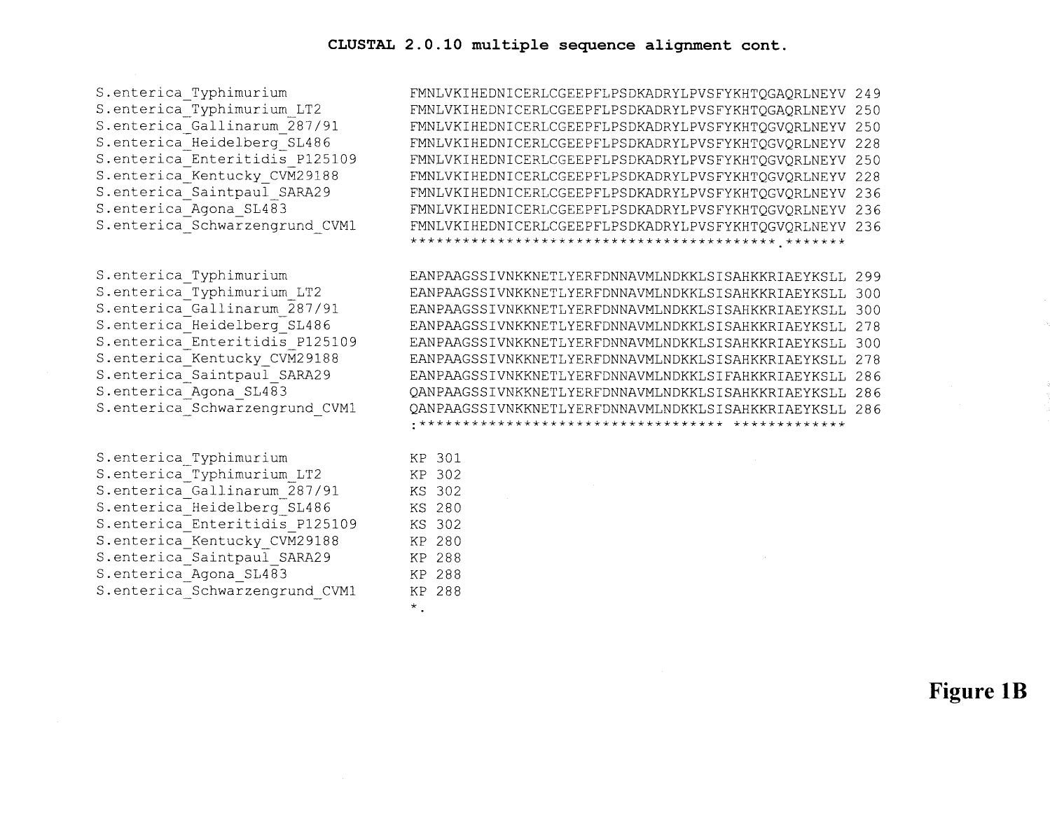

Exemplary Salmonella AvrA homologs are shown in FIGS. 1A-B, including SEQ ID NO: 1 (Salmonella enterica serovar Typhimurium; Genbank Accession No. AAB83970, which is hereby incorporated by reference in its entirety); SEQ ID NO: 2 (Salmonella typhimurium LT2; Genbank Accession No. AAL21745, which is hereby incorporated by reference in its entirety); SEQ ID NO: 3 (Salmonella enterica subsp. enterica serovar Gallinarum str. 287/91; Genbank Accession No. CAR38577, which is hereby incorporated by reference in its entirety); SEQ ID NO: 4 (Salmonella enterica subsp. enterica serovar Heidelberg str. SL486; Genbank Accession No. EDZ24776, which is hereby incorporated by reference in its entirety); SEQ ID NO: 5 (Salmonella enterica subsp. enterica serovar Enteritidis str. P125109; Genbank Accession No. CAR34285, which is hereby incorporated by reference in its entirety); SEQ ID NO: 6 (Salmonella enterica subsp. enterica serovar Kentucky str. CVM29188; Genbank Accession No. EDX43702, which is hereby incorporated by reference in its entirety); SEQ ID NO: 7 (Salmonella enterica subsp. enterica serovar Saintpaulia, strain SARA23; Genbank Accession No. EDZ12687, which is hereby incorporated by reference in its entirety); SEQ ID NO: 8 (Salmonella enterica subsp. enterica serovar Agona strain SL483; Genbank Accession No. ACH48766, which is hereby incorporated by reference in its entirety); and SEQ ID NO: 9 (Salmonella enterica subsp. enterica serovar Schwarzengrund str. CVM19633; Genbank Accession No. ACF92027, which is hereby incorporated by reference in its entirety). These nine sequences shown in FIGS. 1A-B share between 97-100% identity.

Other Salmonella AvrA homologs are also known in the art, including those for strains HI_N05-537, SL317, CVM23701, SL254, CT_{—}02021853, SL480, SL491, SARA29, SL476, CVM29188, CDC 191, and R105P066 (see, e.g., Genbank Accession Nos. ZP_{—}02832444.1, ZP_{—}02697922.1, ZP_{—}02575265.1, ZP_{—}02679382.1, ZP_{—}02352067.1, ZP_{—}02568199.1, ZP_{—}02660958.1, ZP_{—}02704254.1, ZP_{—}02344048.1, ZP_{—}02667530.1, ZP_{—}02671513.1, ZP_{—}02560426.1, ZP_{—}02655988.1, ZP_{—}02683990.1, AAL21745.1, AF250312.1, each of which is hereby incorporated by reference in its entirety).

According to one embodiment, the isolated AvrA proteins or polypeptides include those that are at least about 75 percent identical, more preferably at least about 80 or 85 percent, most preferably at least about 90 or 95 percent identical, to the amino acid sequence of residues 23-302 (301) of SEQ ID NO: 19 (consensus AvrA).

Amino acid sequence homology, or sequence identity, is determined by optimizing residue matches and, if necessary, by introducing gaps as required. See also Needleham et al., J. Mol. Biol. 48:443-453 (1970); Sankoff et al., Chapter One in Time Warps, String Edits, and Macromolecules: The Theory and Practice of Sequence Comparison, Addison-Wesley, Reading, Mass. (1983); and software packages from IntelliGenetics, Mountain View, Calif.; and the University of Wisconsin Genetics Computer Group, Madison, Wis., each of which is hereby incorporated by reference in its entirety. Sequence identity changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. The conservation may apply to biological features, functional features, or structural features. Homologous amino acid sequences are typically intended to include natural polymorphic or allelic and interspecies variations of a protein sequence. In the absence of including gaps and conserved substitutions, identity measures will be at least about 75%, preferably at least about 80%, and more preferably at least about 90% for AvrA homologs.

Fragments of AvrA that possess p53 acetylation capability can also be used to practice the present invention. Fragments can be identified by screening fragments of varying size and structure for the ability to acetylate p53 in an in vitro acetylation assay as described in the accompanying Examples. Exemplary fragments include those missing N-terminal portions of AvrA protein, but possessing C-terminal portions thereof.

Suitable fragments can be produced by several means. Subclones of the gene encoding a known protein can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial or other host cells to yield a smaller protein or polypeptide that can be tested for activity, e.g., as a product capable of acetylating p53.

In another approach, based on knowledge of the primary structure of the protein, fragments of the protein-coding gene may be synthesized using the PCR technique together with specific sets of primers chosen to represent particular portions of the protein. Erlich et al., “Recent Advances in the Polymerase Chain Reaction,” Science 252:1643-51 (1991), which is hereby incorporated by reference in its entirety. These can then be cloned into an appropriate vector for expression of a truncated protein or polypeptide from bacterial or other cells as is well known in the art.

As an alternative, fragments of a protein can be produced by digestion of a full-length protein with proteolytic enzymes like chymotrypsin or Staphylococcus proteinase A, or trypsin. Different proteolytic enzymes are likely to cleave different proteins at different sites based on the amino acid sequence of the particular protein. Some of the fragments that result from proteolysis may be active AvrA polypeptides, and can be screened in vitro for their ability to acetylate p53 as described in the accompanying examples.

Chemical synthesis can also be used to make suitable fragments. Such a synthesis is carried out using known amino acid sequences for the polypeptide being produced. Alternatively, subjecting a full length protein to high temperatures and pressures will produce fragments. These fragments can then be separated by conventional procedures (e.g., chromatography, SDS-PAGE).

Variants may also (or alternatively) be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Other variants include those possessing single or multiple substitutions of one or more domains. A number of variants are identified in the examples, including those exhibiting reduced p53 acetylation and those deficient for de-ubiquitination (see Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). Upon expression of these variants in suitable host cells, activity of the variants can be screened using the methods described herein or in Ye et al., “Salmonella Effector AvrA Regulation of Colonic Epithelial Cell Inflammation by Deubiquitination,” Am. J. Pathol. 171:882-892 (2007), which is hereby incorporated by reference in its entirety. Variants may include one or more conserved substitutions, as identified above.

Other embodiments of these proteins or polypeptide include fusion proteins that are formed, e.g., by an in-frame gene fusion to result in the expression of AvrA protein or polypeptide fragment thereof fused to a second polypeptide, such as an affinity tag for purification or identification, a fluorescent polypeptide for in situ visualization of the fusion protein, or any polypeptides that promote cancer cell uptake of the fusion protein (e.g., antibodies or binding fragments thereof that recognize and bind to cancer cell surface markers).

The proteins or polypeptides used in accordance with the present invention are preferably produced in purified form (preferably at least about 80%, more preferably 90%, pure) by conventional techniques, preferably by isolation from recombinant host cells. In such cases, to isolate the protein, the host cell (e.g., E. coli) carrying a recombinant plasmid is propagated, lysed by sonication, heat, or chemical treatment, and the homogenate is centrifuged to remove bacterial debris. The supernatant is then subjected to sequential ammonium sulfate precipitation. The fraction containing the protein or polypeptide of interest can be subjected to gel filtration in an appropriately sized dextran or polyacrylamide column to separate the proteins. If necessary, the protein fraction may be further purified by HPLC.

Also encompassed by the present invention are isolated nucleic acid molecules encoding the AvrA proteins or polypeptides of the present invention. The isolated nucleic acid molecule can be DNA or RNA, and it can also contain non-naturally occurring nucleic acids.

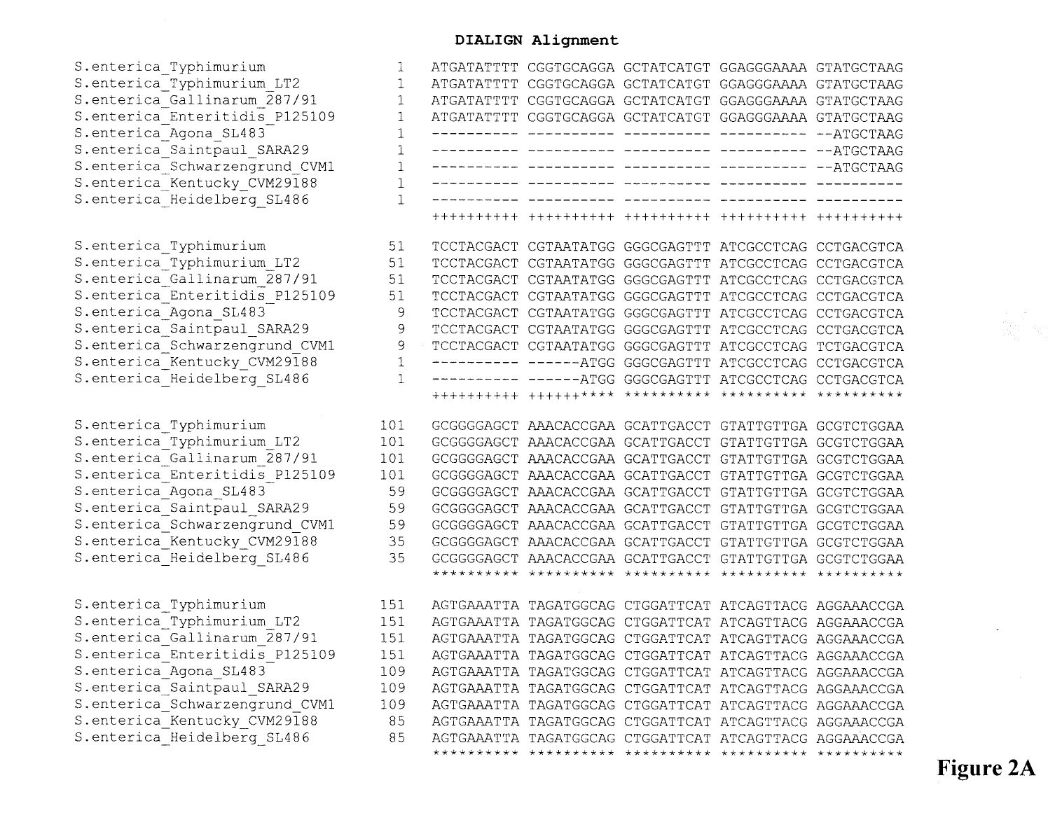

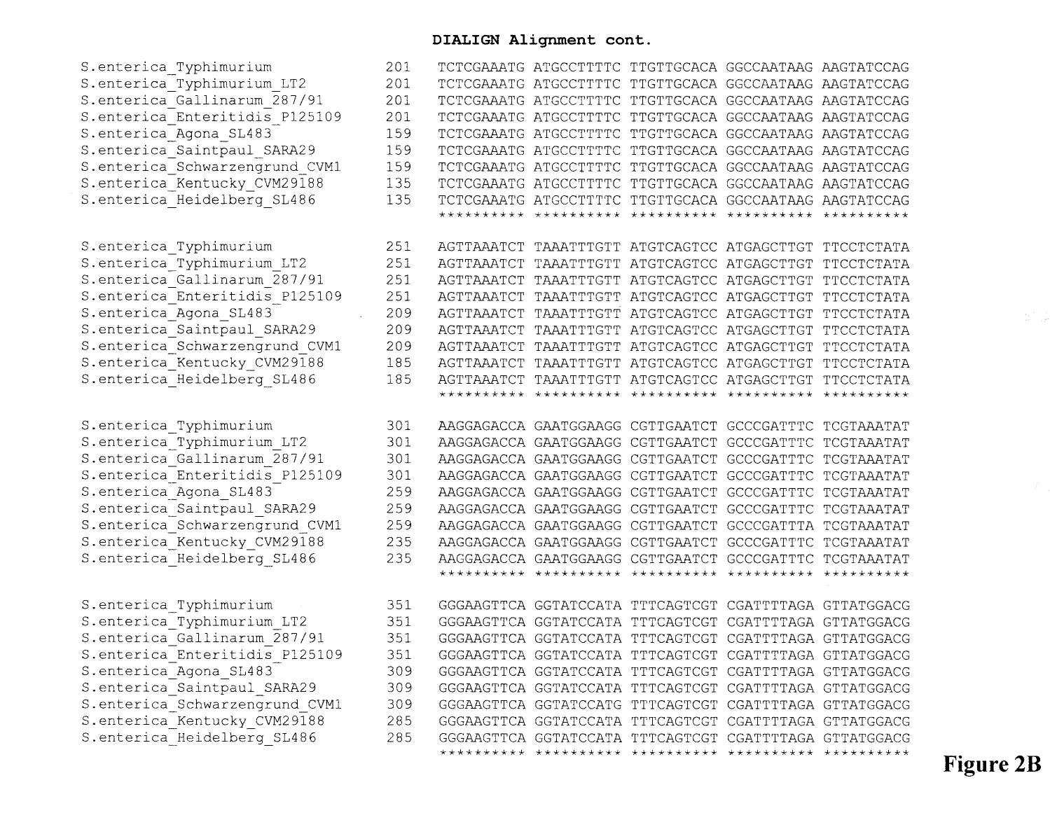

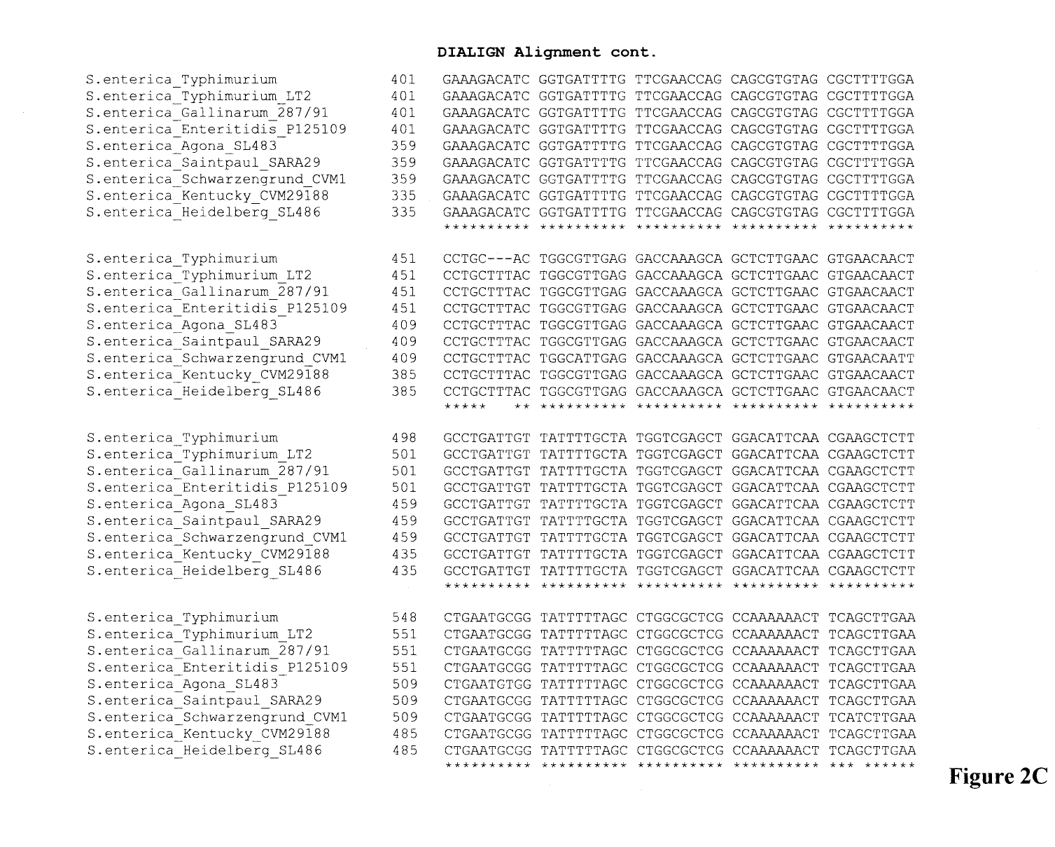

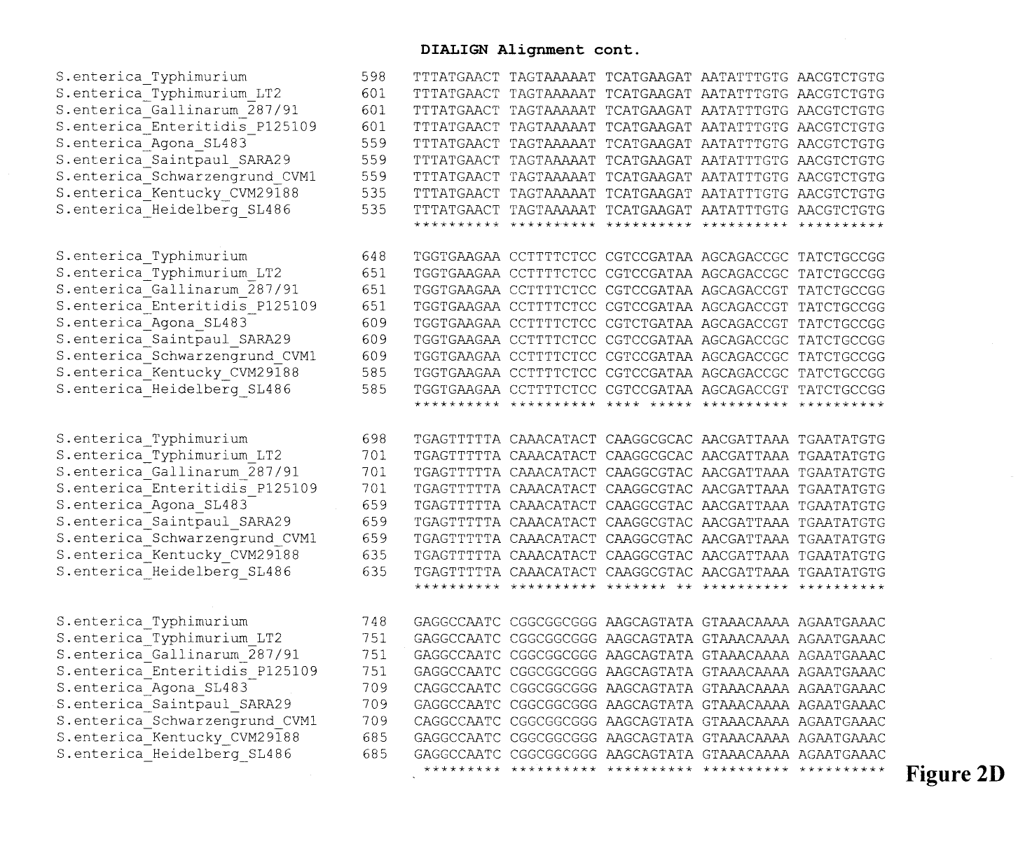

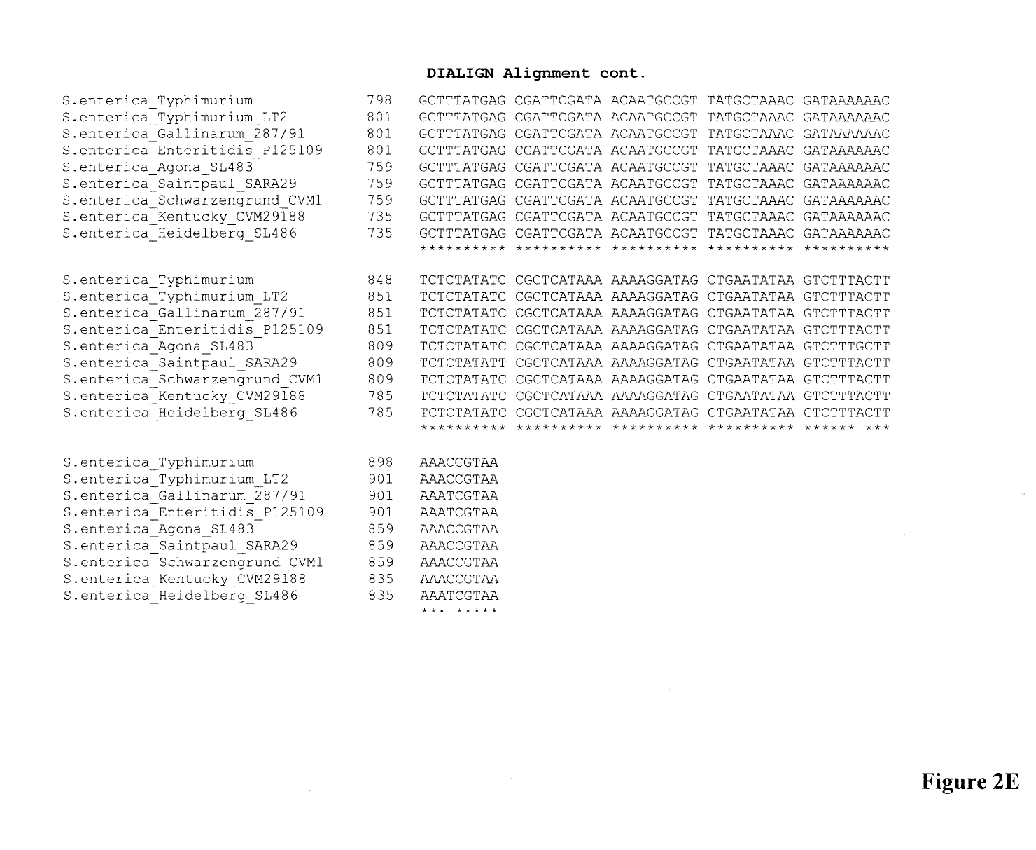

Exemplary DNA molecules encoding AvrA are shown in FIGS. 2A-E, including without limitation, SEQ ID NO: 10 (Salmonella enterica serovar Typhimurium; Genbank Accession No. AF013573, which is hereby incorporated by reference in its entirety); SEQ ID NO: 11 (Salmonella typhimurium LT2; Genbank Accession No. AE006468, which is hereby incorporated by reference in its entirety); SEQ ID NO: 12 (Salmonella enterica subsp. enterica serovar Gallinarum str. 287/91; Genbank Accession No. AM933173, which is hereby incorporated by reference in its entirety); SEQ ID NO: 13 (Salmonella enterica subsp. enterica serovar Heidelberg str. SL486; Genbank Accession No. ABEL01000005, which is hereby incorporated by reference in its entirety); SEQ ID NO: 14 (Salmonella enterica subsp. enterica serovar Enteritidis str. P125109; Genbank Accession No. AM933172, which is hereby incorporated by reference in its entirety); SEQ ID NO: 15 (Salmonella enterica subsp. enterica serovar Kentucky str. CVM29188; Genbank Accession No. ABAK02000001, which is hereby incorporated by reference in its entirety); SEQ ID NO: 16 (Salmonella enterica subsp. enterica serovar Saintpaulia, strain SARA23; Genbank Accession No. ABAN01000004, which is hereby incorporated by reference in its entirety); SEQ ID NO: 17 (Salmonella enterica subsp. enterica serovar Agona strain SL483; Genbank Accession No. CP001138, which is hereby incorporated by reference in its entirety); and SEQ ID NO: 18 (Salmonella enterica subsp. enterica serovar Schwarzengrund str. CVM19633; Genbank Accession No. CP001127, which is hereby incorporated by reference in its entirety). The DNA molecules encoding other homologous AvrA proteins, including those identified above, have been identified in Genbank.

Also encompassed by the present invention are nucleic acid molecules that encode other AvrA homologs that share at least 75 percent identity at the amino acid level, and are capable of hybridizing over substantially their full length to the complement of any one of SEQ ID NOS: 10-18 under stringent hybridization and wash conditions. Exemplary stringent hybridization and wash conditions include, without limitation, hybridization carried out for about 8 to about 20 hours at a temperature of about 42° C. using a hybridization medium that includes 0.9× sodium citrate (“SSC”) buffer, followed by washing for about 5 minutes to about 1 hour with 0.2×SSC buffer at 42° C. Higher stringency can readily be attained by increasing the temperature for either hybridization or washing conditions or decreasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency. Exemplary high stringency conditions include carrying out hybridization at a temperature of about 55° C. up to and including about 65° C. (inclusive of all temperatures in such range) for about 8 up to about 20 hours in a hybridization medium containing 1M NaCl, 50 mM Tris-HCl, pH 7.4, 10 mM EDTA, 0.1% sodium dodecyl sulfate (SDS), 0.2% ficoll, 0.2% polyvinylpyrrolidone, 0.2% bovine serum albumin, and 50 μg/ml E. coli DNA, followed by washing for about 5 minutes to about 1 hour, at about 55° C. up to and including about 65° C. (inclusive of all temperatures in such range) in a 0.2×SSC buffer.

Also encompassed by the present invention are codon-enhanced nucleic acid molecules that have their codons modified to enhance expression in a particular type of host cell during recombinant production and purification thereof.

The preparation of the nucleic acid constructs of the present can be carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Suitable vectors include, but are not limited to, vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et al., “Use of T7 RNA Polymerase to Direct Expression of Cloned Genes,” Gene Expression Technology Vol. 185 (1990), which is hereby incorporated by reference in its entirety), and any derivatives thereof. Several viral systems including murine retrovirus, adenovirus, parvovirus (adeno-associated virus), vaccinia virus, and herpes virus, such as herpes simplex virus and Epstein-Barr virus, and retroviruses, such as MoMLV have been developed as therapeutic gene transfer vectors (Nienhuis et al., Hematology, Vol. 16:Viruses and Bone Marrow, N. S. Young (ed.), pp. 353-414 (1993), which is hereby incorporated by reference in its entirety). Viral vectors provide a more efficient means of transferring genes into cells as compared to other techniques such as calcium phosphate or DEAE-dextran-mediated transfection, electroporation, or microinjection. It is believed that the efficiency of viral transfer is due to the fact that the transfer of DNA is a receptor-mediated process (i.e., the virus binds to a specific receptor protein on the surface of the cell to be infected.) Among the viral vectors that have been cited frequently for use in preparing transgenic mammal cells are adenoviruses (U.S. Pat. No. 6,203,975 to Wilson). In one embodiment of the present invention, a nucleic acid encoding the AvrA protein of the present invention is incorporated into an adenovirus or adeno-associated expression vector.

Once a suitable expression vector is selected, the desired nucleic acid sequence(s) cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, each of which is hereby incorporated by reference in its entirety. The vector is then introduced to a suitable host.

A variety of host-vector systems may be utilized to express the recombinant protein or polypeptide inserted into a vector as described above. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria, viral vectors, either with or without biolistics. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. The promoters can be constitutive or, alternatively, tissue-specific or inducible. In addition, in some circumstances inducible (TetOn) tissue-specific promoters can be used.

According to one embodiment, a cell-specific and/or tumor-specific promoter is used to construct the expression vector encoding AvrA, such that expression thereof can be limited to cancer cells or the tissue in which the tumor to be treated exists. Tumor-specific promoters include, with limitation, DF3 (MUC1), alpha-fetoprotein (AFP), carcinoembryonic antigen (CEA), prostate specific antigen (PSA), tyrosinase, B-myb, and c-erbB2. Cell-specific promoters include, without limitation, endothelial nitric oxide synthase (eNOS) promoter expressed in endothelial cells; vascular endothelial growth factor (VEGF) receptor (flk1) promoter expressed in endothelial cells; insulin promoter expressed in beta cells of the pancreas; promoter of gonadotropin-releasing hormone receptor gene expressed in cells of the hypothalamus; matrix metalloproteinase 9 promoter, expressed in osteoclasts and keratinocytes; promoter of parathyroid hormone receptor expressed in bone cells; or dopamine beta-hydroxylase promoter expressed in noradrenergic neurons. Other tumor-specific promoter can also be used in accordance with the present invention.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. Depending on the vector system and host utilized, any number of suitable transcription and/or translation elements, including constitutive, inducible, and repressible promoters, as well as minimal 5′ promoter elements, enhancers or leader sequences may be used.

In eukaryotic systems, the polyadenylation signal sequence may be selected from any of a variety of polyadenylation signal sequences known in the art. Preferably, the polyadenylation signal sequence is the SV40 late polyadenylation signal sequence. The construct may also include sequences in addition to promoters which enhance expression in the cancer cells or tissues where the tumor resides (e.g., enhancer sequences, introns, etc.).

Typically, when a recombinant host is produced, an antibiotic or other compound useful for selective growth of the transgenic cells only is added as a supplement to the media. The compound to be used will be dictated by the selectable marker element present in the plasmid with which the host was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

The selection marker employed will depend on the target species and/or host or packaging cell lines compatible with a chosen vector.

A nucleic acid molecule encoding the desired product of the present invention (AvrA protein or polypeptide fragment, or fusion protein), a promoter molecule of choice, including, without limitation, enhancers, and leader sequences; a suitable 3′ regulatory region to allow transcription in the host, and any additional desired components, such as reporter or marker genes, can be cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, each of which is hereby incorporated by reference in its entirety.

Once the expression vector has been prepared, it is ready to be incorporated into a host. Recombinant molecules can be introduced into cells by any suitable means including, without limitation, via transformation (if the host is a prokaryote), transfection (if the host is a eukaryote), transduction (if the host is a virus), conjugation, mobilization, or electroporation, lipofection, protoplast fusion, mobilization, particle bombardment, or electroporation, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety.

Suitable hosts include, but are not limited to, bacteria, virus, yeast, and mammalian cells (e.g., human cells, whether as a cell line or primary cell isolates), including, without limitation, whole organisms in need of gene therapy for treatment of cancer.

Accordingly, another aspect of the present invention relates to a method of making a recombinant cell. Basically, this method is carried out by transforming a host with a nucleic acid construct of the present invention under conditions effective to yield transcription of the nucleic acid molecule in the host. Preferably, a nucleic acid construct containing a suitable nucleic acid molecule of the present invention is stably inserted into the genome of the recombinant host as a result of the transformation. Alternatively, the construct can be intentionally used for transient transfection, which results in the loss of the transgene phenotype over time.

As noted above, the present invention contemplates therapeutic administration to a mammalian subject, preferably though not exclusively human subject, of either the AvrA protein or polypeptide, a fusion protein containing the same, or a nucleic acid molecule or expression vector of the present invention. Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, domesticated animals, and animals used in agriculture.

Therapeutic administration thereof can be achieved by any suitable means, but preferably via parenteral (e.g., intravenous, intraarterial, intramuscular, subcutaneous injection, direct injection to tumor site), oral (e.g., dietary), topical, nasal, inhalation, or rectal routes, or via slow releasing microcarriers. According to one embodiment, the pharmaceutical composition is administered directly to a tumor site to be treated. According to another embodiment, the pharmaceutical composition is administered systemically.

Oral, parenteral and intravenous administration are preferred modes of administration. Formulation of the compound to be administered will vary according to the route of administration selected (e.g., solution, emulsion, gels, aerosols, capsule). An appropriate pharmaceutical composition containing the protein or polypeptide or nucleic acid to be delivered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980), which is hereby incorporated by reference in its entirety).

Nanoparticle-based powder formulations are known for inhalation-mediated delivery. Nanoparticles for the purpose of drug delivery are defined as submicron colloidal particles, including both monolithic nanoparticles (nanospheres) in which the drug is adsorbed, dissolved, or dispersed throughout the matrix and nanocapsules in which the drug is confined to an aqueous or oily core surrounded by a shell-like wall. Alternatively, the drug can be covalently attached to the surface or into the matrix. Nanoparticles are made from biocompatible and biodegradable materials such as polymers, either natural (e.g., gelatin, albumin) or synthetic (e.g., polylactides, polyalkylcyanoacrylates), or solid lipids. In the body, the drug loaded in nanoparticles is usually released from the matrix by diffusion, swelling, erosion, or degradation. A number of these types of nanoparticle formulations are known and can be adapted for delivery of AvrA polypeptides or AvrA-encoding nucleic acids of the present invention. Examples of such formulations include, without limitation, those described in U.S. Reissue Pat. No. RE 37,053 to Hanes et al., U.S. Pat. No. 6,503,480 to Edwards et al., U.S. Application Publ. No. 20090297613 to Ringe et al., and U.S. Application Publ. No. 20090312402 to Contag et al.

Therapeutically effective administration of the AvrA protein or polypeptide or fusion protein of the invention typically occurs in doses ranging from 0.1 mg/kg of body weight to 25 mg/kg. In some embodiments, the therapeutically effective dose is 0.3, 1.0, 3, 5, 7.5, 10 and 25 mg/kg. An amount effective to treat cancerous conditions depends upon such factors as the efficacy of the active compounds, the molecular weight of the agent chosen, the nature and severity of the disorders being treated and the weight of the patient. However, a unit dose will normally contain 0.01 to 200 mg, for example 20 to 100 mg, of the compound of the invention. “Unit dose” includes a discrete amount of the pharmaceutical composition comprising a predetermined amount of the active ingredient. In some embodiments, a dose of 1-200 mg of AvrA is injected as a single bolus in a human in need of treatment, including but not limited to a human with cancer. In some embodiments, a dose of 20 to 100 mg is administered. In another embodiment, 1-200 mg of AvrA is administered orally.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally-administrable formulations that are useful include those, which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

In one embodiment, the present invention provides AvrA encapsulated in a polymer or other material that is resistant to acid hydrolysis or acid breakdown. In one embodiment, this formulation provides rapid release of AvrA upon entry into the duodenum. Accordingly, the invention includes a composition containing an AvrA protein or polypeptide or fusion protein and a pharmaceutically-acceptable acid-resistant (“enteric”) carrier. By acid-resistant is meant that the carrier or coating does not dissolve in an acidic environment. An acidic environment is characterized by a pH of less than 7. The acid-resistant carrier is resistant to acids at pH less than about 4.0. Preferably, the carrier does not dissolve in pH 2-3. Most preferably, it does not dissolve in pH of less than 2. In embodiments, the enteric coating is pH-sensitive. The coating dissolves after the pH is greater than 4.0. For example, the coating dissolves in a neutral environment as is encountered in the small intestine, and does not dissolve in an acidic environment as is encountered in the stomach. Alternatively, the enteric coating dissolves when exposed to specific metabolic event such as an encounter with a digestive enzyme that is found in the small intestine. For example, the coating is digested by a pancreatic enzyme such as trypsin, chymotrypsin, or a pancreatic lipase. Enteric coating materials are known in the art, e.g., malic acid-propane 1,2-diol. Cellulose derivatives, e.g., cellulose acetate phthalate or hydroxypropyl methylcellulose phthalate (HPMCP), are also useful in enteric acid-resistant coatings. Other suitable enteric coatings include cellulose acetate phthalate, polyvinyl acetate phthalate, methylcellulose, hydroxypropylmethylcellulose phthalate and anionic polymers of methacrylic acid and methyl methacrylate. Another suitable enteric coating is a water emulsion of ethylacrylate methylacrylic acid copolymer, or hydroxypropyl methyl cellulose acetate succinate (HPMAS). See, e.g., U.S. Pat. Nos. 5,591,433, 5,750,104 and 4,079,125, each of which is hereby incorporated by reference in its entirety. An enteric coating is designed to resist solution in the stomach and to dissolve in the neutral or alkaline intestinal fluid. See also coatings described in Wilding et al., “Targeting of Drugs and Vaccines to the Gut,” Pharmac. Ther. 62:97-124 (1994), which is hereby incorporated by reference in its entirety. In another embodiment, lyophilized, particulate AvrA mixed with bicarbonate (as buffer) is coated with Eudragit S100, L30D or L 100-44 according to the manufacturer's instructions (Evonik Industries).

In another embodiment, the formulations of the invention are those used successfully with lactase (see U.S. Pat. No. 6,008,027 to Langner et al., which is hereby incorporated by reference in its entirety). In this embodiment, gelatin capsules are filled with 50-90% lyophilized AvrA, the remaining capacity being filled with stabilizing dessicants such as silicon oxide, silicon dioxide or microcrystalline cellulose and bicarbonate buffer. The capsules are enterically coated with Eudragit polymer (Evonik Industries) or polyvinyl acetate phthalate (Sureteric, Colorcon) and vacuum dried prior to use. Similarly, diastase has been formulated with Eudragits RS 100 and cellulase acetate phthalate coatings for enteric use, and the present invention provides novel formulations that resemble these but contain AvrA instead of diastase (Vyas et al., “Enteric Spherules of Diastase in Enzyme Preparations, J. Microencapsulation 8: 447-454 (1991), which is hereby incorporated by reference in its entirety).

For delivery of nucleic acid-based therapies, a number of different approaches can be employed. For example, naked DNA can be delivered in accordance with U.S. Pat. No. 6,831,070 to German et al., which is hereby incorporated by reference in its entirety. Alternatively, the nucleic acid can be formulated into a delivery vehicle, such as the chitosan hexamer-PEI vector described in Ouji et al., J. Biosci. Bioeng. 94(1):81-3 (2002), which is hereby incorporated by reference in its entirety, or chitosan-DNA nanoparticles containing an AAV expression vector as described in Chen et al., World J Gastroenterol 10(1):112-116 (2004), which is hereby incorporated by reference in its entirety. Alternatively, DNA can be incubated with an inert carbohydrate polymer (dextran) to which a positively charged chemical group (DEAE, for diethylaminoethyl) has been coupled. The DNA sticks to the DEAE-dextran via its negatively charged phosphate groups. Other polymer-based delivery vehicles can be used, including poly(ester amine) (Arote et al., “Biodegradable poly(ester amine)s for Gene Delivery Application,” Biomed. Mater. 4:044102(DOI) (2009); Arote et al., “A Biodegradable Poly(ester amine) Based on Polycaprolactone and Polyethyleneimine as a Gene Carrier,” Biomaterials 28(4):735-744 (2007); Guo et al., “Synthesis of Novel Biodegradable Poly(ester amine) (PEAs) Copolymer Based on Low-Molecular Weight Polyethyleneimine for Gene Delivery,” Int. J. Pharmaceutics 379(1):82-89 (2009), each of which is hereby incorporated by reference in its entirety) and poly(amido amine) (Piest et al., “Novel Poly(amido amine)s with Bioreducible Disulfide Linkages in Their Diamine Units Structure Effects and in vitro Gene Transfer Properties,” J. Controlled Release 132(3):e12-13 (2008); Lin et al., “Novel Bioreducible Poly(amido amine)s for Highly Efficient Gene Delivery,” Bioconjugate Chem. 18(1):138-145 (2007), each of which is hereby incorporated by reference in its entirety). These large DNA-containing particles stick in turn to the surfaces of cells, which are thought to take them in by a process known as endocytosis.

The AvrA proteins or polypeptides and encoding nucleic acids of the present invention can also be administered or used in combination with other known cancer therapies including, without limitation, surgery, immunotherapies, chemotherapies, and radiation therapies. These combination therapies can be administered according to any effective dosing schedule, which may involve co-administration at distinct or the same sites, or at different times.

According to one embodiment, the AvrA protein or polypeptide (or encoding nucleic acid) is administered in combination with a chemotherapeutic agent. Exemplary chemotherapeutic agents include, without limitation, various toxins (e.g., diphtheria, ricin, and cholera toxin); alkylating agents (e.g., cisplatin, carboplatin, oxaloplatin, mechlorethamine, cyclophosphamide, chorambucil, nitrosureas); anti-metabolites (e.g., methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g., vincristine, vinblastine, vinorelbine), podophyllotoxin (e.g., etoposide, teniposide), taxanes (e.g., paclitaxel, docetaxel); topoisomerase inhibitors (e.g., irinotecan, topotecan, amasacrine, etoposide phosphate); antitumor antibiotics (e.g., dactinomycin, doxorubicin, epirubicin, and bleomycin); ribonucleotides reductase inhibitors; antimicrotubules agents; and retinoids.

According to one embodiment, the AvrA protein or polypeptide (or encoding nucleic acid) is administered in combination with a radiotherapeutic agent. Exemplary radiotherapeutic agents include, without limitation, any nuclide emitting radioactive ray usable for the cancer treatment, including x-ray, gamma-ray, electron beam, photon, alpha-particle and neutron, etc. The above nuclide is exemplified by ¹³¹I, ⁶⁰Co, ⁵⁷Co, ¹⁹²Ir, ¹⁶⁶Ho, ³²P, ⁴⁸V, ¹⁹⁸Au, ^{99m}Tc, ¹²⁵I, ¹⁶⁵Dy, ¹⁸⁸Re, ¹⁶⁹Er, ¹⁵³Sm, ⁹⁰Y, ¹⁰⁹Pd, and ⁸⁹Sr. Such radioactive rays are preferably in complex with a carrier such as chitosan to prevent leakage from the tumor lesion to the surrounding tissue.

According to one embodiment, the AvrA protein or polypeptide (or encoding nucleic acid) is administered in combination with an immunotherapeutic agent. Exemplary immunotherapeutic agents include, without limitation, interleukin-1 (IL-1), IL-2, IL-4, IL-5, IL-0, IL-7, IL-10, IL-12, IL-15, IL-18, CSF-GM, CSF-G, IFN-γ, IFN-α, TNF, TGF-β, FLT-3 ligand, CD40 ligand, and antibody-mediated delivery agents.

According to another embodiment, the AvrA protein or polypeptide (or encoding nucleic acid) is administered in combination with a nutlin-based therapy of the type described in PCT Application Publ. No. WO 2008/106507 to Chen et al.; Hori et al., “Nutlin-3 Enhances Tumor Necrosis Factor-related Apoptosis-inducing Ligand (TRAIL)-induced Apoptosis Through Up-regulation of Death Receptor 5 (DR5) in Human Sarcoma HOS Cells and Human Colon Cancer HCT116 Cells,” Canc. Lett. 287(1):98-108 (2010); Myachi et al., “Restoration of p53 Pathway by Nutlin-3 Induces Cell Cycle Arrest and Apoptosis in Human Rhabdomyosarcoma Cells,” Clin. Cancer Res. 15:4077-4084 (2009); Van Maerken et al., “Antitumor Activity of the Selective MDM2 Antagonist Nutlin-3 Against Chemoresistant Neuroblastoma With Wild-Type p53,” J. Nat'l Cancer Inst. 101(22):1562-1574 (2009), each of which is hereby incorporated by reference in its entirety.

Thus, the compounds of the invention and the other pharmacologically active agents may be administered to a patient simultaneously, sequentially or in combination. If administered sequentially, the time between administrations of each individual drug generally varies from 0.1 to about 48 hours. More preferably, the time between administrations varies from 4 hours and 24 hours. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

### EXAMPLES

The following examples are provided to illustrate embodiments of the present invention but are by no means intended to limit its scope.

### Materials and Methods for Examples 1-9

Bacterial Strains and Growth Condition: Bacterial strains used in this study included Salmonella typhimurium wild-type strain ATCC14028, E. coli F18 and non-pathogenic Salmonella mutant strain PhoP^{c} (Miller et al., “Constitutive Expression of the PhoP Regulon Attenuates Salmonella Virulence and Survival within Macrophages,” J. Bacteriol 172:2485-2490 (1990), which is hereby incorporated by reference in its entirety), PhoP^{c} AvrA−, and PhoP^{c} AvrA−/AvrA+, PhoP^{c} AvrA+, wild-type Salmonella SL1344 (SB300), and its AvrA mutant strainSB1117. PhoP^{c} AvrA+ was generated by over-expressing AvrA gene in pWSK29 low-copy plasmid (see Table 1 below). Non-agitated microaerophilic bacterial cultures were prepared as previously described (Sun et al., “Bacterial Activation of beta-Catenin Signaling in Human Epithelia,” Am. J. Physiol. 287:G220-227 (2004), which is hereby incorporated by reference in its entirety).

TABLE 1
Types of Salmonella Strains of the Present Invention

| Name | Description |
|---|---|
| Salmonella 14028s | Wild-type S. typhimurium |
| Salmonella AvrA⁺ | 14028s with AvrA overexpressing in pWSK29 |
| | low-copy plasmid |
| SL1344(SB300) | Wild-type Salmonella 1344 strain |
| SB1117 | SL 1344 with mutant AvrA |
| PhoP^{C} | Non-pathogenic complex regulator mutant |
| PhoP^{C}AvrA⁻ | Non-pathogenic complex regulator mutant |
| | without AvrA |
| PhoP^{C}AvrA⁻/AvrA⁺ | PhoP^{C}AvrA− complemented with AvrA in plasmid |
| PhoP^{C} AvrA⁺ | 14028s AvrA− |

AvrA Point Mutation: The avrA gene is from wild-type S. typhimurium strain SL3201. AvrA point-mutations were generated by using QuikChange II Site-Directed Mutagenesis Kit (Stratagene, La Jolla, Calif.). DNA sequencing was performed by Functional Genomics Center of University of Rochester.

AvrA and Its Mutant Protein Purification. Salmonella full length gene AvrA and single point mutants AvrA(C186A), AvrA(R180G), AvrA(C179A), AvrA(E142A), or AvrA(H123A) were cloned into N-terminal glutathione S-transferase (GST) fused Vector pEGX-4T2 (Invitrogen) and transformed into E. coli strain BL21 (DE3). Affinity purification was performed using a glutathione-Sepharose resin (Amersham Bioscience, Piscataway, N.J., USA) as previously described (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety).

Cell Culture: Human embryonic kidney 293 cells, Caco2-BBE, HT29Cl.29A, epithelial HeLa cells, and MEFs were maintained in DMEM supplemented with 10% FCS, penicillin-streptomycin and L-glutamine. Human colonic epithelial HCT116 cells and the p53−/− knockout cells were cultured in McCoy's 5A medium supplemented with 10% (vol/vol) fetal bovine serum. The rat small intestinal IEC-18 cell line was grown in DMEM (high glucose, 4.5 g/L) containing 5% (vol/vol) fetal bovine serum, 0.1 U/ml insulin, 50 μg/ml streptomycin, and 50 U/ml penicillin.

Streptomycin Pre-treated Mouse Model: Animal experiments were performed by using specific-pathogen-free female C57BL/6 mice (Taconic) that were 6-7 weeks old as previously described (Duan et al., “Beta-Catenin Activity Negatively Regulates Bacteria-Induced Inflammation,” Laboratory Investigation; A Journal of Technical Methods and Pathology 87:613-624 (2007), which is hereby incorporated by reference in its entirety). Water and food were withdrawn 4 h before oral gavage with 7.5 mg/mouse of streptomycin (100 μA of sterile solution or 100 μA of sterile water in control). Afterwards, animals were supplied with water and food ad libitum. Twenty hours after streptomycin treatment, water and food were withdrawn again for 4 hours before the mice were infected with 1×10⁷ CFU of S. Typhimurium (100 μl suspension in HBSS) or treated with sterile HBSS (control) by oral gavage as previously described (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). At indicated time after infection, mice were sacrificed and tissue samples from the intestinal tracts were removed for analysis. For the survival rate of mice post Salmonella typhimurium infected, mice (n=20 per group) were infected with 1×10⁸ CFU Salmonella typhimurium strain 14028s (WT) with insufficient AvrA expression or 14028s with AvrA overexpression (WTAvrA+) and observed for 7 days. If a mouse showed indication that it had aspirated fluid or significant body weight loss (10% or more), and did not die immediately, the mouse was humanely euthanized. The protocol was approved by the University of Rochester University Committee on Animal Resources (UCAR).

Mouse Colonic Epithelial Cells: Mouse colonic epithelial cells were collected by scraping the mouse colon including proximal and distal regions. Cells were sonicated in lysis buffer (1% Triton X-100, 150 mM NaCl, 10 mM Tris pH 7.4, 1 mM EDTA, 1 mM EGTA, pH 8.0, 0.2 mM sodium ortho-vanadate, protease inhibitor cocktail) and the protein concentration was determined (BioRad). β-actin was used as the loading control for all Western blots. Villin, an accepted marker of epithelial cells (Grone et al., “A Marker of Brush Border Differentiation and Cellular Origin in Human Renal Cell Carcinoma,” Am. J. Pathol. 124:294-302 (1986), which is hereby incorporated by reference in its entirety), was used as the control for epithelial cell protein content in all Western blots.

Transient Transfections: Transient transfections were performed with LipofectAMINE2000 (Invitrogen, San Diego, Calif.) in accordance with the manufacturer's instructions. Briefly, 6×10⁵ HEK293 cells were seeded on 60 mm dishes overnight before co-transfection 4 μg cMyc tagged AvrA or C186A with 2 μg GFP tagged p53, or 2 μg HA-tagged p53, respectively. DNA was mixed with the liposome reagent at a ratio of 1:1 before adding to cells. After a 24-hour transfection, proteins were extracted with RIPA buffer (50 μM Tris-Hcl, PH8.0 with 150 mM sodium chloride, 1.0% NP-40, 0.5% sodium deoxycholate, 0.1% SDS) for immunoblotting.

Immunoblotting: Mouse colonic epithelial cells were collected by scraping from mouse colon including proximal and distal regions (Duan et al., “Beta-Catenin Activity Negatively Regulates Bacteria-Induced Inflammation,” Laboratory Investigation; A Journal of Technical Methods and Pathology 87:613-624 (2007), which is hereby incorporated by reference in its entirety). Cells were sonicated in lysis buffer (1% Triton X-100, 150 mM NaCl, 10 mM Tris pH 7.4, 1 mM EDTA, 1 mM EGTA pH 8.0, 0.2 mM sodium ortho-vanadate, protease inhibitor cocktail). The protein concentration was measured using BioRad Reagent (BioRad, Hercules, Calif., USA). Cultured cells were rinsed twice in ice-cold HBSS, lysed in protein loading buffer (50 mM Tris, pH 6.8, 100 mM dithiothreitol, 2% SDS, 0.1% bromophenol blue, 10% glycerol), and sonicated. Equal amounts of protein were separated by SDS-polyacrylamide gel electrophoresis, transferred to nitrocellulose, and immunoblotted with primary antibodies. The following antibodies were used: monoclonal mouse anti-c-Myc or HA (Santa Cruz Biotechnology Inc., Santa Cruz, Calif., U.S.A.), monoclonal mouse anti-GFP, monoclonal mouse anti-p53, monoclonal mouse anti-Bax, anti-Bcl-2, anti-p21(Santa Cruz), anti-acetylated p53(373), p53(373/382), or phosphorylated p53 ser9 (Cell Signal, Beverly, Mass.), anti-acetyl-Lysine anti-acetylated p53(320) (Upstate, Temecula, Calif., U.S.A.), monoclonal mouse anti-p14ARF, monoclonal mouse anti-MDM2 (Abcam, Cambridge, Mass., U.S.A.), or anti-β-actin (Sigma-Aldrich, Milwaukee, Wis., U.S.A.).

Co-immunoprecipitation Assay: Cells were rinsed twice in ice-cold HBSS and lysed in cold immunoprecipitation buffer (1% Triton X-100, 150 mM NaCl, 10 mM Tris.HCl, pH 7.4, 1 mM EDTA, 1 mM EGTA, pH 8.0, 0.2 mM sodium orthovanadate) containing protease inhibitor cocktail. Samples were precleared with protein A-agarose. Precleared lysates were then incubated with indicated antibody. Co-immunoprecipitation samples were separated by SDS-polyacrylamide gel electrophoresis, and transferred onto a nitrocellulose membrane (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). Membrane blots were probed with anti-c-myc or anti-p53 antibody and visualized by enhanced chemiluminescence.

Immunofluorescence: Colonic tissues from the proximal and distal portion of the colon were freshly isolated and embedded in paraffin wax after fixation with 10% neutral buffered formalin. Immunohistochemistry was performed on paraffin-embedded sections (1 μm) of mouse colons. Tissue samples or cultured cells were processed as described previously (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). Cells or tissues were mounted with SlowFade (SlowFade® AntiFade Kit, Molecular Probes) followed by a coverslip, and the edges were sealed to prevent drying. Specimens were examined with a Leica SP5 Laser Scanning confocal microscope.

In vitro AvrA Transacetylase Assays: For the cell-free AvrA transacetylase assay, purified wild-type p53 protein (Santa Cruz, sc4246) was used as the substrate. The wild-type AvrA and AvrA mutant proteins were purified from Escherichia coli strain BL21(DE3). 50 microliter reactions contained 10 μl 5× reaction buffer (250 mM Tris, pH8.0, 50% glycerol, 0.5 mM EDTA, 5 mM DTT), 5 μl 0.1 mM Acetyl-CoA (Sigma), 5 μl 100 μg/ml P53, and 20 μg of different mutant proteins. Transacetylase PCAF (Upstate) was used as a positive control. The reaction mixture was incubated at 30° C. for 30 minutes. The reactions were stopped by the addition of an equal volume of a SDS-gel sample buffer, denatured, then, followed immunoblot for detection.

p53 Transcriptional Activation ELISA: HCT116 p53−/− cells were grown in 12-well plates in triplicate. The cells were transfected with different plasmids. The plasmid groups include a pFC-p53 plasmid, a control plasmid pRL-TK, a p53-Luc-cis reporter plasmid cotransfected with plasmid pRL-TK (Stratagene, La Jolla, Calif.), a c-myc-AvrA cotransfected a p53-Luc-cis reporter and a pRL-TK plasmid, or a p53-Luc-cis reporter, a pFC-p53 plasmid, and a pRL-TK using LipofectAMINE. The control plasmid pRL-TK contains a Renilla reporter gene driven by the thymidine kinase promoter (Promega, Madison, Wis.), using LipofectAMINE (Invitrogen). After transfection for 24 hours, cells were lysed, and luciferase activity was determined using the Dual Luciferase Reporter Assay System (Promega) with a TD-20/20 luminometer (Turner Designs, Sunnyvale, Calif.). Firefly luciferase activity was normalized to Renilla luminescence activity, and then activity was expressed as relative units.

Cell Cycle: Cell cycle was analyzed by DNA Content (Propidium Iodide staining) IEC-18 cells were colonized by the indicated bacterial strains at 37° C. for 30 min, washed, incubated for 4 h in DMEM with Gentamicin. Forskolin (50 μM) treatment was used as a positive control. Cells were collected, washed twice with Hank's, and fixed for 1 hour using 1 ml methanol pre-chilled at −20° C. Cell samples were resuspended in 400 μl PI/RNase staining buffer (BD, Franklin Lakes, N.J., U.S.A.) at room temperature for 10 minutes, then subjected to flow cytometry in a Beckman Coulter Epics XL MCL and 50,000 cells were collected for cell cycle analysis.

Salmonella-induced Human IL-8 Secretion: HCT116 p53−/− or p53+/+ cells were cultured in DMEM, followed by Salmonella-containing HBSS (1.6×10¹⁰ bacteria/ml) for 30 min, washed 3 times in HBSS, and incubated at 37° C. for 6 hours. Cell supernatants were removed and assayed for IL-8 by ELISA in 96-well plates as described previously (McCormick et al., “Salmonella typhimurium Attachment to Human Intestinal Epithelial Monolayers: Transcellular Signalling to Subepithelial Neutrophils,” J. Cell. Biol. 123:895-907 (1993), which is hereby incorporated by reference in its entirety).

Real Time Quantitative PCR Analysis of IL-8. Total RNA was extracted from epithelial cell monolayers using TRIzol reagent (Invitrogen, Carlsbad, Calif.). The RNA integrity was verified by electrophoresis gel. RNA reverse transcription was done using the iScript cDNA synthesis kit (Bio-Rad, Hercules, Calif.) according to the manufacturer's directions. The RT cDNA reaction products were subjected to quantitative real-time PCR using the MyiQ single-color real-time PCR detection system (Bio-Rad) and iQ SYBR green supermix (Bio-Rad) according to the manufacturer's directions. IL-8 cDNA was amplified by using primers to the human IL-8 gene that are complementary to regions in exon 1 (5′-TGCATAAAGACATACTCCAAACCT, SEQ ID No: 20) and overlapping the splice site between exons 3 and 4 (5′-AATTCTCAGCCCTCTTCAAAAA, SEQ ID No: 21). All expression levels were normalized to the GAPDH levels of the same sample, using forward (5-CTTCACCACCATGGAGAAGGC, SEQ ID No: 22) and reverse (5′-GGCATGGACTGTGGTCATGAG, SEQ ID No: 23) primers for GAPDH. Percent expression was calculated as the ratio of the normalized value of each sample to that of the corresponding untreated control cells. All real-time PCR reactions were performed in triplicate. All PCR primers were designed using Lasergene software (DNAStar, Madison, Wis.).

Statistical Analysis: Data are expressed as mean±SD. Differences between two samples were analyzed by Student's t test. Differences between groups were analyzed using ANOVA (SAS 9.2 version). P-values of 0.05 or less were considered significant.

### Example 1

### Salmonella but not TNFα Increases Acetylation of p53

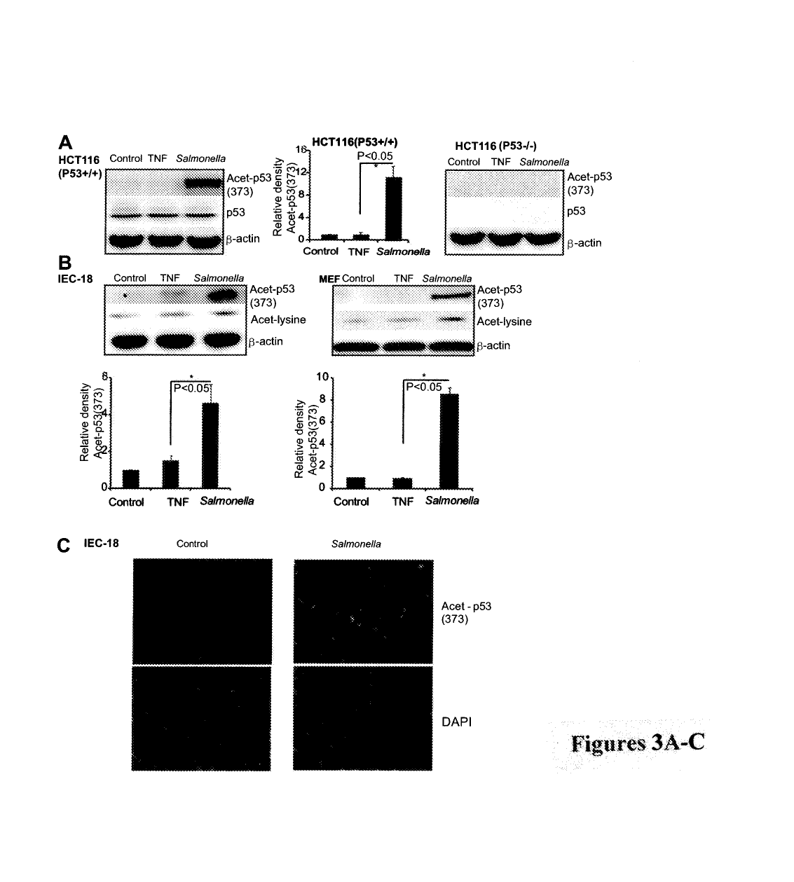

To determine whether Salmonella plays a role in modulating the p53 pathway, epithelial HCT116 cells were colonized with wild-type (WT) Salmonella 14028 or TNF-α, a proinflammatory cytokine Salmonella increased the p53 acetylation in host cells after bacterial colonization for only 1 hour (FIG. 3A, HCT116 p53+/+). In contrast, TNFα had less effect on p53 acetylation. HCT116 p53−/− p53 knockout cells were used as a negative control. There is no total p53 expression in HCT116 p53−/− cells (FIG. 3A, HCT116 p53−/−). The response of the human colonic epithelial cells HT29 C1.19A or Caco-2 BBE was also investigated, and similar changes of Salmonella-induced p53 acetylation were found (FIGS. 11A-B). These commonly used, transformed human colonic epithelial cells possess a mutated p53.

To confirm these findings, the normal epithelial IEC-18 cells, a non-transformed cell line, and mouse embryonic fibroblast cells (MEF) which possess wild-type p53 protein were also examined. As shown in FIG. 3B, Salmonella colonization increased the acetylated form of p53 at position 373. TNFα had no effect on the acetylation of p53 at position 373. The total acetylated lysine (ace-lysine) was also determined (FIG. 3B). Whereas Salmonella increased acetylation of p53 at position 373, total acetylated lysine was only slightly increased (FIG. 3B, IEC-18 and MEF). Using immunofluorescence staining, the location of acetylated p53 in the intestinal epithelial cells was investigated (FIG. 3C). There is little acetylated p53 staining in the control cells without treatment. Salmonella colonization induced the nuclear acetylated p53. Overall, these data indicate that the change of p53 acetylation was induced by Salmonella.

### Example 2

### AvrA Expression Increases Acetylated p53

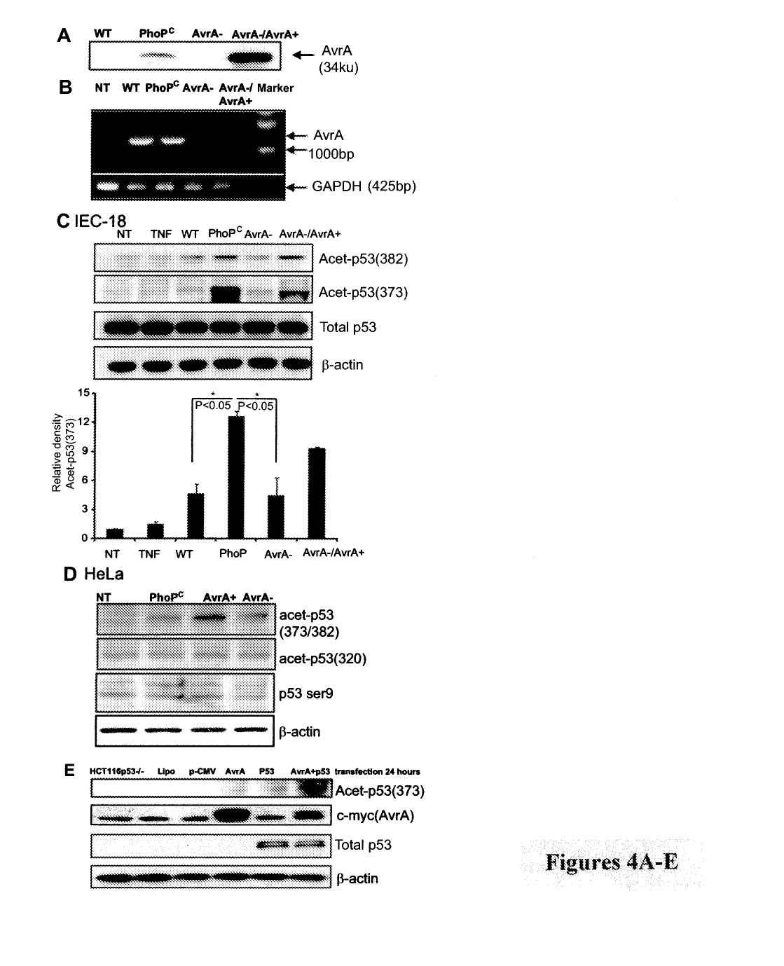

The next inquiry concerned whether bacterial effector AvrA is involved in the acetylation of p53, which was confirmed using bacterial strains sufficient or deficient in AvrA. Western blot assay in FIG. 4A shows the AvrA protein levels in strains used in these studies. The wild-type strain ATCC14028 (WT) has very low AvrA protein expression. PhoP^{c} is a mutant strain derived from WT ATCC 14028 with sufficient AvrA protein expression (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). The parental PhoP^{c} AvrA mutant (AvrA−), or the AvrA complementary strain (PhoP^{c} AvrA−/AvrA+) were used in previous studies (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). This system allowed for examination of the cellular function of AvrA and exclusion of other bacterially-induced effects on the host. After bacterial colonization, AvrA expression in the host cells was determined by PCR (FIG. 4B). In IEC-18 cells, parental PhoP^{c} strain colonization increased acetylated p53 whereas cells treated with PhoP^{c} AvrA-bacteria and WT strain ATCC 14028, which has decreased levels of AvrA protein expression (Moll et al., “The MDM2-p53 Interaction,” Mol Cancer Res 1:1001-1008 (2003); Harper et al., “The p21 Cdk-Interacting Protein Cip1 is a Potent Inhibitor of G1 Cyclin-dependent Kinases,” Cell 75:805-816 (1993), each of which is hereby incorporated by reference in its entirety), displayed reduced levels of acetylated p53. In cells colonized with PhoP^{c} AvrA−/AvrA+, the complementary AvrA expression increased the acetylated forms of p53 at amino acid positions 382 and 373 (FIG. 4C, IEC18).

Salmonella-induced p53 acetylation was also tested in different cell lines of mouse and human origin. Human epithelial HeLa cells were treated with AvrA-sufficient or -deficient Salmonella strains for 6 hours, and the AvrA+ strain increased p53 acetylation at 373 and 382 sites (FIG. 4D, HeLa). Acetylation at amino acid position 320 was not changed by Salmonella colonization. There is no change of the phosphorylated p53 at ser9 either. Using transient transfection of a wild-type pCMV-myc-AvrA plasmid or a mutant AvrAC186A plasmid into human colonic epithelial cells, the role of AvrA in enhancing p53 acetylation in host cells was determined. Intestinal epithelial Caco2BBE and HT29Cl.19A cells were transfected with either the pCMV-myc-AvrA or AvrA mutant C186A plasmid (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety) for 24 hours. Total cell lysates were analyzed for protein levels by immunoblot. The results confirm that overexpression of AvrA increased p53 acetylation in epithelial cells (FIGS. 4E, 12A). The AvrA mutant C186A had less effect on p53 acetylation compared to the wild-type AvrA. In the MEF cells, parental PhoP^{c} strain colonization increased acetylated p53 at position 373, whereas PhoP^{c} AvrA− bacteria decreased acetylated p53 at amino acid 373 (FIG. 12B). The complementary AvrA expression in PhoP^{c}AvrA-−/AvrA+restored p53 acetylation. Taken together, these data demonstrate that Salmonella protein AvrA induces the acetylation of p53 at positions 373 and 382 in the host epithelial cells and fibroblasts.

### Example 3

### AvrA Displays a Physical Interaction with p53

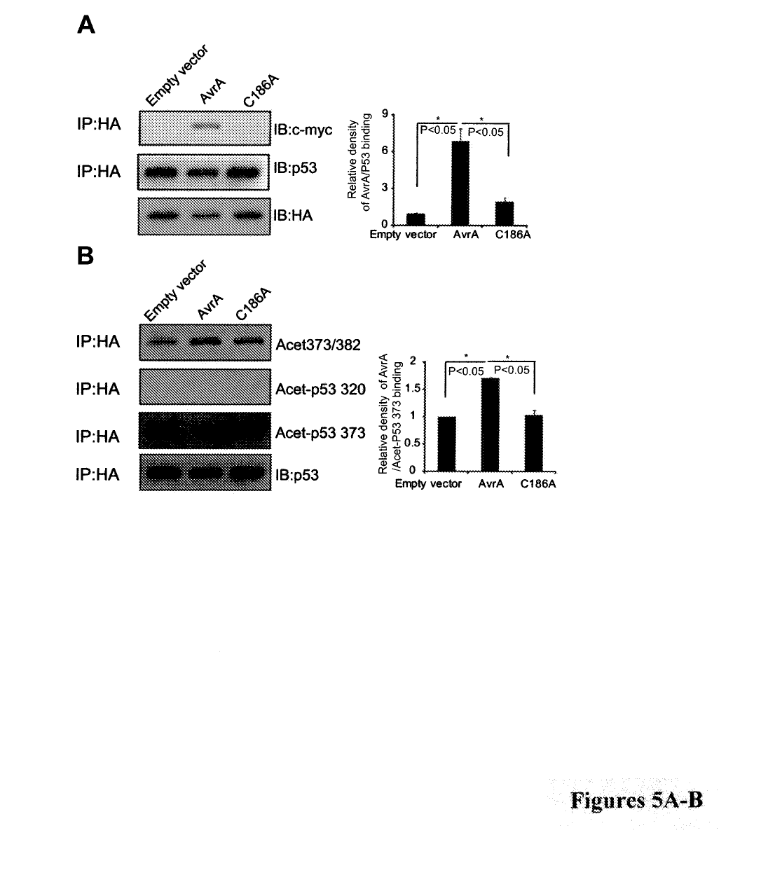

To establish whether the increased p53 acetylation was occurring through physical interaction between AvrA and p53, it was initially determined whether AvrA can form a complex with p53 in epithelial cells. A c-myc-AvrA plasmid DNA was co-transfected with the hemagglutinin epitope YPYDVPDYA (HA tag, SEQ ID NO: 24) p53 for 24 hours, then immunoprecipitated with HA. Immunoblot with HA clearly showed that AvrA interacts with p53, whereas less p53 was observed to be bound to the AvrA mutant C186A which lacks de-ubiquitin (DUB) activity (FIG. 5A). Densitometry data further showed that AvrA mutant C186A significantly reduced the p53/AvrA binding. In addition, green fluorescent protein (GFP)-p53 was co-transfected with c-myc-AvrA for 24 hours. Immunoblot with c-myc also showed AvrA interaction with p53 (FIG. 13). The role of AvrA in targeting the acetylated p53 at positions 373 and 382 during Salmonella infection was further examined. C-myc-tagged AvrA was cotransfected with HA-p53, and then HA beads were used to perform pull-down assays. The acetylation of p53 at sites 382 and 373 was detected by Western blot (FIG. 5B). However, there was no association of AvrA with acetylated p53 at position 320 (FIG. 5B). Moreover, densitometry data indicated that AvrA mutant C186A significantly decreased the p53 acetylation (FIG. 5B).

### Example 4

### AvrA Displays a Physical Interaction with p53 Acetylation

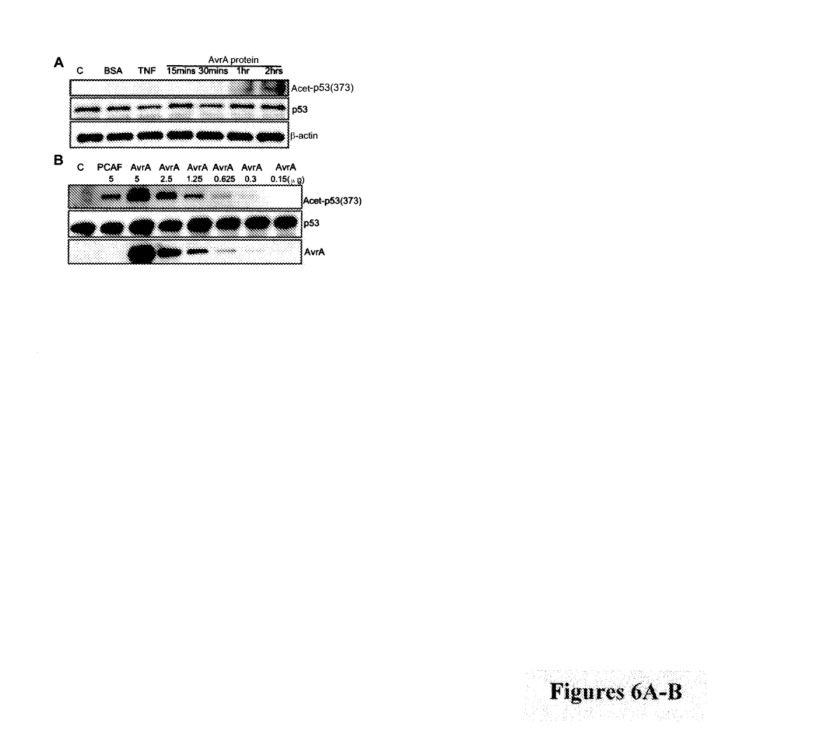

Studies on other effector proteins have shown that bacterial effector may function independent of the TTSS as a protease (Hsu et al., “Structure of the Cyclomodulin Cif from Pathogenic Escherichia coli,” J Mol Biol 384:465-477 (2008), which is hereby incorporated by reference in its entirety). The AvrA wild-type protein was purified and tested for its effects on p53 acetylation. HCT116 cells were directly treated with the AvrA protein in the culture media. It was found that AvrA protein treatment was able to increase p53 acetylation in the intestinal epithelial cells, whereas TNFα or bovine serum albumin (BSA) proteins did not change p53 acetylation (FIG. 6A). In a cell free system for the AvrA transacetylase assay, purified wild-type p53 protein was used as the substrate. Transacetylase p300-CBP-associated factor (PCAF) was used as a positive control. As shown in FIG. 6B, AvrA was able to acetylate p53 in the cell free system. AvrA was used at different concentrations and mixed with p53 in the reaction buffer (FIG. 6B). The level of acet-p53 enhanced with increasing AvrA concentrations in the cell-free system, indicating that AvrA's effect on p53 acetylation is dose-dependent.

### Example 5

### AvrA Increases p53 Transcriptional Activity and Modifies Target Genes of p53

P53 protein has a relatively short half-life of about 20 minutes. In unstressed cells, p53 usually exists in a latent form, and at low levels. P53 is activated rapidly in response to stress including microbial infection. P53 activation involves posttranslational modification, including ubiquitination and acetylation (Kruse et al., “SnapShot: p53 Posttranslational Modifications,” Cell 133:930-930 e931 (2008); Vousden et al., “Live or Let Die: The Cell's Response to p53,” Nature Reviews 2:594-604 (2002), each of which is hereby incorporated by reference in its entirety). Under various types of stress, the transcriptional activity of p53 increases dramatically.

The effects of AvrA on the regulation of p53 transcriptional activity were then investigated. HCT116p53−/− cells were transfected with a pFC-p53 positive control plasmid (Fc), a p53-Luc-cis reporter plasmid (P53), an internal control pRL-TK plasmid (TK) or a pCMV-myc-AvrA. As shown in FIG. 7A, single pFC-p53, TK, or p53+TK cotransfection had no effect on the transcriptional activity, while AvrA and p53 co-transfection significantly increased p53 transcription activity to a level comparable to the positive control group with the p53 and pFC-p53 co-transfection (FIG. 7A). This data indicated that the expression of bacterial AvrA was able to increase p53 transcriptional activity in intestinal epithelial cells.

The effect of AvrA on the p53's target gene expression in host cells was further investigated. Human epithelial cells were colonized with the bacterial strains PhoP^{C}, PhoP^{c} with AvrA overexpression (PhoP^{C}AvrA+), and AvrA mutation. The protein levels of downstream target genes of p53 including p21, BAX, p14ARF, and bcl-2, were investigated by Western blot (FIG. 7B). Etoposide and Staurosporine (STS) were used as controls. The resulting data showed Etoposide, acting primarily in the G2 and S phases of the cell cycle, increased p53 expression, whereas STS, a nonselective protein kinase inhibitor that has been shown to induce apoptosis, decreased p53 expression. With AvrA overexpression in the PhoP^{c}AvrA+ strain, the protein levels of p21 and BAX were decreased (FIG. 7B). AvrA expression had no effect on p14ARF and BCL-2 expression. MDM2, a p53-specific E3 ubiquitin ligase, is the principal cellular antagonist of p53, acting to limit the p53 growth-suppressive function in unstressed cells (Wertz et al., “De-ubiquitination and Ubiquitin Ligase Domains of A20 Downregulate NF-kappaB Signalling,” Nature 430:694-699 (2004), which is hereby incorporated by reference in its entirety). Interestingly, cells colonized with bacterial strains PhoP^{C}, PhoP^{c} with AvrA overexpression, and AvrA mutation all had elevated MDM2 expression compared to the cells without treatment or cells treated with Etoposide or STS. PhoP^{C}AvrA with AvrA overexpression showed a slightly less MDM2 expression compared to the parental PhoP^{C} group. p21, which mediates the p53-dependent cell cycle G1 phase arrest in response to a variety of stress stimuli was also examined (Harper et al., “The p21 Cdk-Interacting Protein Cip1 is a Potent Inhibitor of G1 Cyclin-Dependent Kinases,” Cell 75:805-816 (1993), which is hereby incorporated by reference in its entirety). Cells colonized with bacterial PhoP^{c} were able to increase p21 expression, as did the Etoposide treatment, whereas PhoP^{c}AvrA+decreased p21 expression.

### Example 6

### Salmonella Infection Increases the Acetylation of p53 in a Mouse Model

A streptomycin pretreated mouse model of Salmonella infection (Barthel et al., “Pretreatment of Mice with Streptomycin Provides a Salmonella Enterica Serovar Typhimurium Colitis Model that Allows Analysis of Both Pathogen and Host,” Infect. Immun. 71:2839-2858 (2003), which is hereby incorporated by reference in its entirety) was used to confirm the in vitro findings. One concern for the in vivo study is confirmation of the bacterial colonization and invasion ability in the intestinal epithelial cells. To address the location by which S. typhimurium infects the intestine, infected colon tissues for Salmonella lipopolysaccharide were stained by immunofluorescence. As shown in FIG. 8B, S. typhimurium bacteria were found in the mucosa. This localization indicates that Salmonella invaded intestinal epithelium. Mice colonic epithelial cells were collected post Salmonella infection. It was found that wild-type Salmonella increased the acetylation of p53 post infection for 2 hours and was persistent for over 6 hours (FIG. 8A). In addition, immunofluorescence staining showed increased acetylated p53 (red staining) in the epithelial cell nuclei after Salmonella colonization (mouse colons FIG. 8C). Since the AvrA mutant generated from the SL14028 genetic background was not available, wild-type Salmonella SB300 and its AvrA mutant strain SB1117 (AvrA−) were used to test the effects of AvrA in regulating p53 acetylation in vivo. Salmonella SB300 with AvrA expression significantly increased acetylated p53, and SB1117 (AvrA−) did not change the level of p53 acetylation (FIG. 8D). Because the acetylation of p53 at the C-terminus is related to p53 stability (Kruse et al., “SnapShot: p53 Posttranslational Modifications,” Cell 133:930-930 e931 (2008), which is hereby incorporated by reference in its entirety), the level of total p53, related regulator MDM2, and target gene p21 was also examined. Total p53 decreased in Salmonella infected mice over 4 days. The MDM2 and p21 expression were increased by SB300 with AvrA, whereas the AvrA deficient SB1117 did not change the expression of MDM2 and p21 (FIG. 8D).

### Example 7

### AvrA Expression Induces Cell Cycle Arrest

P53 pathway activation directly increases p21 for cell cycle regulation and leads to the cell cycle arrest in both G0 and G1 (Harper et al., “Inhibition of Cyclin-Dependent Kinases by p21,” Mol. Biol. Cell. 6:387-400 (1995), which is hereby incorporated by reference in its entirety). The cell physiological function of AvrA in regulating the cell cycle in intestinal epithelial cells was further investigated. Intestinal epithelial IEC-18 cells were infected with bacteria. The experimental groups include C: normal IEC-18 cells without any treatment; F: positive control forskolin; T: TNFα; S: wild-type Salmonella ATCC14028; P: PhoP^{C} with AvrA expression, A−: AvrA−; A+: AvrA− restored with AvrA in plasmid; SB300: wild-type Salmonella SL1344; SB1117: AvrA mutant from SL1344. Comparing PhoP^{c} to PhoP^{c} AvrA− or SB300 to SB1117 with deficient AvrA, it was found that AvrA expression significantly increased cell numbers at G0/G1 phase and decreased cell numbers at G2/M phase (FIG. 9A, *AvrA-sufficient group vs. AvrA deficient group, p<0.01). These data indicate that AvrA expression was able to induce cell cycle arrest at G0/G1 in the host cells.

### Example 8

### p53−/− Cells are Less Susceptible to Salmonella Infection

Based on the preceding Examples, it was expected that p53 plays a key role in response to Salmonella stress signal and inflammation. Therefore, without p53 expression in the cells, Salmonella loses a target protein and p53−/− cells could be less susceptible to the bacterial stimulation. The effect of p53 expression on the IL-8 secretion in cells colonized with WT Salmonella was assessed. As shown here (FIG. 9B), there is a significant difference of IL-8 secretion in the cell lines with different status of p53 expression. HCT116 cells with p53 significantly increased the IL-8 protein secreted in the cell media after salmonella colonization for 6 hours. In contrast, the p53−/− HCT116 cells had less inflammatory IL-8 protein secretion after Salmonella colonization (FIG. 9B). In addition, IL-8 real-time PCR showed that the IL-8 mRNA was significantly lower with the p53−/− cells comparing to the p53+/+HCT116 cells with Salmonella colonization (FIG. 9B). These data indicate that intestinal epithelial cells without p53 are less susceptible to Salmonella infection.

### Example 9

### AvrA Overexpression in the Wild-type Salmonella Decreases the Mice Survival Rate

Since the inhibitory function of AvrA on inflammation in the host is observed at early stages of infection (Liao et al., “Salmonella Type III Effector AvrA Stabilizes Cell Tight Junctions to Inhibit Inflammation in Intestinal Epithelial Cells,” PLoS ONE 3:e2369 (2008); Ye et al., “Salmonella Effector AvrA Regulation of Colonic Epithelial Cell Inflammation by Deubiquitination,” Am. J. Pathol. 171:882-892 (2007), each of which is hereby incorporated by reference in its entirety), it was also investigated whether the AvrA inhibitory effect on inflammatory response is beneficial or harmful for the host. Mice were infected with WT Salmonella typhimurium strain 14028s (WT) with insufficient AvrA expression or WT 14028s with AvrA overexpression (WTAvrA+). The survival rate of mice post Salmonella typhimurium infected for over 7 days is shown in FIG. 9C. At day 2, the survival percentage of the WT group is 100%, whereas the WTAvrA+ group is about 60%. Overall, the mice infected with WT S. typhimurium strain 14028s (WT) with insufficient AvrA expression survived longer than the mice with the 14028s with AvrA overexpression (WTAvrA+) (FIG. 9C). This data indicates that AvrA overexpression renders Salmonella highly virulent, resulting in more severe infection and decreased overall survival even after 7 days of infection.

### Discussion of Examples 1-9

In the preceding Examples, it is demonstrated that Salmonella infection increases p53 acetylation via the acetyltransferase activity of the Salmonella effector protein AvrA. Functionally, AvrA expression increased p53 transcriptional activity and induced cell cycle arrest at the G0/G1 phase. In addition, bacterial AvrA expression increased p53 acetylation in intestinal epithelial cells in a mouse model. The present invention also provides direct evidence and a mechanism for how a bacterial protein interferes with host responses via p53 acetylation in intestinal epithelial cells using both in vitro and in vivo systems.

Experimental data also indicate that Salmonella infection is able to increase the expression of MDM2, an E3 ligase for p53 which promotes the ubiquitination and proteasomal degradation of p53. Global microarray analysis on the mouse mucosa infected with Salmonella further showed that Salmonella significantly activated the p53 pathway and its target genes (see Table 2 below). These p53 target genes respond to a variety of stress signals that impact cellular homeostatic mechanisms that monitor DNA replication, chromosome segregation, and cell division (Vogelstein et al., “Surfing the p53 Network,” Nature 408:307-310 (2000), which is hereby incorporated by reference in its entirety). P53 was first shown to be degraded through ubiquitination by the human papilloma virus E6-associated cellular protein E6AP (Scheffner et al., “The HPV-16 E6 and E6-AP Complex Functions as a Ubiquitin-Protein Ligase in the Ubiquitination of p53,” Cell 75:495-505 (1993), which is hereby incorporated by reference in its entirety). E6AP efficiently ubiquitinated and degraded p53 in order to replicate in the host. Taken together, these studies demonstrate that bacteria and viruses exploit the p53 pathway to modulate the host response.

TABLE 2
Microarray Analysis of the Transcriptional Responses of p53 Target
Genes Post Salmonella typhimurium SB300 Infection in Mouse Colon

| Gene Name | Accession No.^{†} | Fold Change | P Value |
|---|---|---|---|
| p21(Pak1) | NM_011035 | 1.45 | 0.017 |
| Mdm2 | NM_010786 | 1.2 | 0.012 |
| EGF2 | NM_207655 | 1.64 | 0.007 |
| Ccnd1 | NM_007631 | 1.48 | 0.012 |
| GML | AJ242831 | 53.22 | 0.0003 |
| ActA1 | NM_009606 | 1.94 | 0.18 |
| Bcl2l1 | NM_009743 | 1.46 | 0.002 |
| Gadd45b | NM_008655 | 1.22 | 0.05 |
| Gadd45g | NM_011817 | 2.63 | 0.01 |
| 14-3-3 σ | AF152893 | 1.51 | 0.08 |
| IRF-5 | NM_012057 | 1.21 | 0.002 |
| Bak1 | NM_007523 | 1.32 | 0.003 |

^{†}Each of the above-listed Accession Nos. is hereby incorporated by reference in its entirety.

AvrA point-mutations at positions 123, 142, 179, 186 (key amino acid sites), and 180 (non-specific amino acid site) were generated to investigate the relative contributions of the potential catalytic residues in AvrA function. Whereas the point-mutations of AvrA at positions 180 and 123 did not change the acetyltransferase activity, point-mutations of AvrA at amino acid sites 186, 142, and 179 sites reduced its acetyltransferase activity (FIG. 14). Although protein sequence and domain prediction can help to determine the potential catalytic amino acid (Nakamura “Isolation of p53-Target Genes and their Functional Analysis,” Cancer Science 95:7-11 (2004), which is hereby incorporated by reference in its entirety), it is not 100% accurate. These data indicate that AvrA could be an acetyltransferase with multiple protease domains and one amino acid point-mutation may not be enough to completely abolish the acetyltransferase activity of AvrA. Moreover, recent study indicated a different initiation codon for AvrA translation (Du et al., “Selective Inhibition of Type III Secretion Activated Signaling by the Salmonella Effector AvrA,” PLoS Pathog 5:e1000595 (2009), which is hereby incorporated by reference in its entirety). The initiation codon in the constructed plasmid may affect the activity of AvrA protein in vitro.

AvrA at site C186 is the key amino acid for the deubiquitinase activity of AvrA (Ye et al., “Salmonella Effector AvrA Regulation of Colonic Epithelial Cell Inflammation by Deubiquitination,” Am. J. Pathol. 171:882-892 (2007), which is hereby incorporated by reference in its entirety). Experimental data demonstrate that AvrA mutant C186A changed the physical binding of AvrA and p53 (FIG. 5). C186A also partially abolished the effect of AvrA on reduction of total p53 protein (FIGS. 7A-B). Data from AvrA point mutation experiments and cell-free system assays indicate that different key amino acids of AvrA contribute to its different enzyme activities. The amino acid at position 186 may primarily control the deubiquitinase activity, whereas positions 142 and 179 regulate primarily acetyltransferase activity. AvrA enzymatically modifies diverse target proteins, including IκBα, β-catenin (Yamaguchi et al., “p53 Acetylation is Crucial for its Transcription-Independent Proapoptotic Functions,” J. Biol. Chem. 284:11171-11183 (2009), which is hereby incorporated by reference in its entirety), MKK7 (Du et al., “Selective Inhibition of Type III Secretion Activated Signaling by the Salmonella Effector AvrA,”PLoS Pathog 5:e1000595 (2009), which is hereby incorporated by reference in its entirety), and p53. Hence, like other deubiquitylating enzymes and acetyltransferases, AvrA appears to act on multiple substrates. As noted above, because AvrA has dual roles (as a deubiquitinase and as an acetyltransferase) in regulation of the signaling pathways in host cell, it is believed that this accounts for its different effects in cancer cells and normal cells.

AvrA has multiple effects on p53 including physical binding, acetylation, and cell cycle modulation. Some of these findings are consistent with a study of the EBV immediate-early protein BZLF1, which had numerous effects on p53 posttranslational modification (Hsu et al., “Structure of the Cyclomodulin Cif from Pathogenic Escherichia coli,” J. Mol. Biol. 384:465-477 (2008), which is hereby incorporated by reference in its entirety). Infection of cells with this BZLF1 vector increased the level of cellular p53 but prevented the induction of p53-dependent cellular target genes, such as p21 and MDM2. BZLF1-expressing cells displayed increased p53 phosphorylation at multiple residues, and increased acetylation at lysine 320 and lysine 382. It is also demonstrated that Salmonella infection increases p53 acetylation. However, while some findings of the present invention are similar, data indicate that AvrA has no effect on the acetylation of p53 at lysine 320. Although the AvrA deficient Salmonella strains are able to reduce p53 acetylation, they cannot completely abolish p53 acetylation. This indicates that other bacterial proteins may also participate in the modification of p53.

It is recognized that using in vitro cell culture can present some limitations. Those limitations include: 1) the use of transformed cell lines that may not necessarily reflect behavior of normal epithelial cells; 2) technical difficulties in performing biological assays beyond 48 hours because cells become confluent; 3) the potential uncontrolled bacterial growth over 24 hours which may damage cells non-specifically even with extensive washing in the presence of antibiotics; and 4) A transient transfection system does not fully mimic the TTSS system which normally delivers bacterial effectors into the host cell. Therefore, the in vivo role of AvrA was assessed using mouse models.

Based on the current findings, it can be concluded that Salmonella uses bacterial effectors including AvrA to increase the acetylation of p53 (FIG. 10). Bacterial effectors are injected by the Salmonella TTSS into the intestinal epithelial cells. Once in the host cells, AvrA may target p53 and act as a transacetylase to induce acetylation of p53, thus activating the p53 pathway and increasing the downstream target genes such as cyclin-dependent kinase inhibitor p21^{CIP1/WAF1/SDI1} (p21). By acting on its target genes, in normal cells AvrA induces intestinal epithelial cell cycle arrest, inhibits apoptosis, and allows the intestinal epithelial cells to survive (FIG. 10). Eventually, AvrA inhibits the host's inflammatory responses and induces severe infection in the host. The cell cycle arrest is one of the biological impacts induced by Salmonella AvrA. AvrA's impacts on intestinal epithelial cell apoptosis, proliferation, and inhibition of inflammation in vivo have been described elsewhere (Liao et al., “Salmonella Type III Effector AvrA Stabilizes Cell Tight Junctions to Inhibit Inflammation in Intestinal Epithelial Cells,” PLoS ONE 3:e2369 (2008); Liu et al., “Salmonella Regulation of Intestinal Stem Cells Through the Wnt/Beta-Catenin Pathway,” FEBS Lett. 594(5):911-916 (2010); and Ye et al., “Salmonella Effector AvrA Regulation of Colonic Epithelial Cell Inflammation by Deubiquitination,” Am. J. Pathol. 171:882-892 (2007), each of which is hereby incorporated by reference in its entirety).

Bacteria play a key role in intestinal homeostasis (Asfaha et al., “Persistent Epithelial Dysfunction and Bacterial Translocation After Resolution of Intestinal Inflammation,” Am. J. Phys. 281:G635-644 (2001); Skinn et al., “Citrobacter Rodentium Infection Causes iNOS-Independent Intestinal Epithelial Dysfunction in Mice,” Can. J. Physiol Pharmacol. 84:1301-1312 (2006); and Wu et al., “A Human Colonic Commensal Promotes Colon Tumorigenesis via Activation of T Helper Type 17 T Cell Responses,” Nat. Med. 15:1016-1022 (2009), each of which is hereby incorporated by reference in its entirety). Although the p53 tumor suppressor has been extensively studied, the preceding Examples demonstrate the unexpected effect of AvrA on p53 acetylation and its implication on disease states such as cancer.

### Example 10

### In vivo Tumor Model

Human tumor cells will be transplanted into SCID mice either under the skin or into the organ type in which the tumor originated. The tumor will be allowed to grow for 8 weeks, and then AvrA will be administered directly to the tumor site, alone, and in combination with a selected chemotherapeutic agent or radiation therapy. Efficacy of the AvrA and combination therapies will be assessed against control mice receiving direct injection of a buffered saline.

### Example 11

### In vivo Tumor Model

Human tumor cells will be transplanted into SCID mice either under the skin or into the organ type in which the tumor originated. The tumor will be allowed to grow for 8 weeks, and then naked DNA (contain an AvrA coding region under control of suitable constitutive promoter) will be administered directly to the tumor site in an anion lipophilic transfection medium. Efficacy of the gene therapy, alone, and in combination with a selected chemotherapeutic agent or radiation therapy will be assessed against control mice receiving direct injection of a buffered saline.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. For example, it is expressly intended that all combinations of those elements and/or method steps which perform substantially the same function in substantially the same way to achieve the same results are within the scope of the invention. Moreover, it should be recognized that elements and/or method steps shown and/or described in connection with any disclosed form or embodiment of the invention may be incorporated in any other disclosed or described or suggested form or embodiment.

1. A method for inhibiting cancer cell proliferation comprising:
introducing into a cancer cell (i) an isolated AvrA protein or polypeptide fragment thereof or (ii) a nucleic acid molecule encoding the isolated AvrA protein or polypeptide fragment, wherein said introducing is effective for inhibiting cell cycle progression of the cancer cell.

2. The method of claim 1, wherein the AvrA protein comprises an amino acid sequence that is at least about 90% identical to SEQ ID NO: 19.

3-4. (canceled)

5. The method of claim 1, wherein the AvrA protein or polypeptide fragment is administered.

6. The method of claim 1, wherein the nucleic acid molecule is administered.

7. The method of claim 6, wherein the nucleic acid molecule is present in an expression vector comprising a promoter operable in mammalian cells, which promoter is operably coupled to the nucleic acid molecule.

8. The method of claim 6, wherein the nucleic acid molecule encodes an AvrA protein that is at least about 90% identical to SEQ ID NO: 19.

9. (canceled)

10. The method according to claim 1, wherein the cancer cell is present in a solid tumor.

11. The method according to claim 1, wherein the cancer cell is metastatic cancer cell or circulating leukemia or lymphoma cell.

12. A method of treating a patient for cancer comprising:
administering to a patient having cancer a therapeutically effective dose of (i) an isolated AvrA protein or polypeptide fragment thereof, or (ii) a nucleic acid molecule encoding the AvrA protein or polypeptide fragment.

13. The method of claim 12, wherein the AvrA protein comprises an amino acid sequence that is at least about 90% identical to SEQ ID NO: 19.

14-15. (canceled)

16. The method of claim 12, wherein the AvrA protein or polypeptide fragment is administered.

17. The method of claim 16, wherein the AvrA protein or polypeptide fragment is administered at a dose ranging from 0.1 mg to 10 mg of said protein or polypeptide per kg of said patient's body weight.

18. The method of claim 16, wherein the isolated AvrA protein or polypeptide fragment is present in a composition.

19. The method of claim 12, wherein the nucleic acid molecule is administered.

20. The method of claim 19, wherein the nucleic acid molecule is present in an expression vector comprising a promoter operable in mammalian cells, which promoter is operably coupled to the nucleic acid molecule.

21. The method of claim 19, wherein the nucleic acid molecule encodes an AvrA protein that is at least about 90% identical to SEQ ID NO: 19.

22. (canceled)

23. The method of claim 12, wherein said administering is carried out parenterally, orally, topically, intranasally, rectally, or via slow releasing microcarriers.

24. The method of claim 12, wherein the cancer is a solid tumor cancer.

25. The method of claim 24, wherein the solid tumor cancer is selected from the group of sarcomas, carcinomas, and gliomas.

26. The method of claim 12, wherein the cancer is a leukemia or lymphoma.

27. The method of claim 12, wherein the method further comprises:
administering to the patient an immunotherapeutic agent, chemotherapeutic agent or radiation therapeutic agent in an amount effective to treat the cancer.

28-32. (canceled)

33. A pharmaceutical composition comprising:
a pharmaceutically acceptable carrier;
a therapeutically effective amount of an isolated AvrA protein or polypeptide fragment thereof; and
a therapeutically effective amount of an immunotherapeutic agent, chemotherapeutic agent, or radiation therapeutic agent.

34. The pharmaceutical composition of claim 33, wherein the AvrA protein comprises an amino acid sequence that is at least about 90% identical to SEQ ID NO: 19.

35-36. (canceled)

37. The pharmaceutical composition of claim 33, wherein the AvrA protein or polypeptide fragment is administered.
